(19) United States
(12) Patent Application Publication
(10) Pub. No.: US 2004/0074491 A1
(43) Pub. Date: Apr. 22, 2004

(54) Delivery methods and devices for implantable bronchial isolation devices

(76) Inventors: Michael Hendricksen, Reedwood City, CA (US); Ronald R. Hundertmark, San Mateo, CA (US); David R. Tholfsen, San Francisco, CA (US); Peter M. Wilson, Killingworth, CT (US); Antony J. Fields, San Francisco, CA (US)

Correspondence Address: Stephanie L. Seidman, Heller Ehrman White & McAuliffe LLP, 7th Floor, 4350 La Jolla Village Drive, Suite 700, San Diego, CA, 92122-1246, US

(21) Appl. No.: 10/645,473

(22) Filed: Aug. 20, 2003

Related U.S. Application Data
(63) Continuation-in-part of application No. 09/797,910, filed on Mar. 2, 2001, now Pat. No. 6,694,979.
(63) Continuation-in-part of application No. 10/270,792, filed on Oct. 10, 2002.
(60) Provisional application No. 60/405,418, filed on Aug. 21, 2002.

(51) Int. Cl. B05B 7/00; A61M 11/00

(52) U.S. Cl. 128/200.19

(57) ABSTRACT

Disclosed are various devices and methods for delivering bronchial isolation devices into the lung of a patient. In accordance with one aspect of the invention, there is disclosed an apparatus for deploying a bronchial device in a bronchial passageway in a lung of a patient. The apparatus comprises a flexible shaft having an inner lumen and a distal end adapted to engage the bronchial isolation device. The apparatus further comprises a wire slidably disposed in the inner lumen and a housing movably coupled to the distal end of the shaft and configured to receive the bronchial device. The wire is connected to the housing to produce relative movement between the housing and the shaft to deploy the bronchial device from the housing.

### REFERENCE TO PRIORITY DOCUMENT

This application claims priority of co-pending U.S. Provisional Patent Application Serial No. 60/405,418 entitled “Delivery Methods And Devices For Implantable Bronchial Isolation Devices”, filed Aug. 21, 2002. Priority of the aforementioned filing date is hereby claimed, and the disclosure of the Provisional Patent Application is hereby incorporated by reference in its entirety.

This application is a continuation-in-part of the following co-pending patent applications: (1) U.S. patent application Ser. No. 09/797,910, entitled “Methods and Devices for Use in Performing Pulmonary Procedures”, filed Mar. 2, 2001; and (2) U.S. patent application Ser. No. 10/270,792, entitled “Bronchial Flow Control Devices and Methods of Use”, filed Oct. 10, 2002. The aforementioned applications are hereby incorporated by reference in their entireties.

### BACKGROUND OF THE INVENTION

1. Field of the Invention

This invention relates generally to methods and devices for use in performing pulmonary procedures and, more particularly, to procedures for treating lung diseases.

2. Description of the Related Art

Certain pulmonary diseases, such as emphysema, reduce the ability of one or both lungs to fully expel air during the exhalation phase of the breathing cycle. Such diseases are accompanied by chronic or recurrent obstruction to air flow within the lung. One of the effects of such diseases is that the diseased lung tissue is less elastic than healthy lung tissue, which is one factor that prevents full exhalation of air. During breathing, the diseased portion of the lung does not fully recoil due to the diseased (e.g., emphysematic) lung tissue being less elastic than healthy tissue. Consequently, the diseased lung tissue exerts a relatively low driving force, which results in the diseased lung expelling less air volume than a healthy lung.

The problem is further compounded by the diseased, less elastic tissue that surrounds the very narrow airways that lead to the alveoli, which are the air sacs where oxygen-carbon dioxide exchange occurs. The diseased tissue has less tone than healthy tissue and is typically unable to maintain the narrow airways open until the end of the exhalation cycle. This traps air in the lungs and exacerbates the already-inefficient breathing cycle. The trapped air causes the tissue to become hyper-expanded and no longer able to effect efficient oxygen-carbon dioxide exchange.

In addition, hyper-expanded, diseased lung tissue occupies more of the pleural space than healthy lung tissue. In most cases, a portion of the lung is diseased while the remaining part is relatively healthy and, therefore, still able to efficiently carry out oxygen exchange. By taking up more of the pleural space, the hyper-expanded lung tissue reduces the amount of space available to accommodate the healthy, functioning lung tissue. As a result, the hyper-expanded lung tissue causes inefficient breathing due to its own reduced functionality and because it adversely affects the functionality of adjacent healthy tissue.

Lung reduction surgery is a conventional method of treating emphysema. However, such a conventional surgical approach is relatively traumatic and invasive, and, like most surgical procedures, is not a viable option for all patients.

Some recently proposed treatments for emphysema or other lung ailments include the use of devices that isolate a diseased region of the lung in order to modify the air flow to the targeted lung region or to achieve volume reduction or collapse of the targeted lung region. According to such treatments, one or more bronchial isolation devices are implanted in airways feeding the targeted region of the lung. The bronchial isolation device regulates airflow through the bronchial passageway in which the bronchial isolation device is implanted. The bronchial isolation devices can be, for example, one-way valves that allow flow in the exhalation direction only, occluders or plugs that prevent flow in either direction, or two-way valves that control flow in both directions.

The following references describe exemplary bronchial isolation devices: U.S. Pat. No. 5,954,766 entitled “Body Fluid Flow Control Device”; U.S. patent application Ser. No. 09/797,910, entitled “Methods and Devices for Use in Performing Pulmonary Procedures”; and U.S. patent application Ser. No. 10/270,792, entitled “Bronchial Flow Control Devices and Methods of Use”. The foregoing references are all incorporated by reference in their entirety and are all assigned to Emphasys Medical, Inc., the assignee of the instant application.

The bronchial isolation device can be implanted in a target bronchial passageway using a delivery catheter that is guided with a guidewire that is placed through the trachea (via the mouth or the nasal cavities) and through the target location in the bronchial passageway. A commonly used technique is to perform what is known as an “exchange technique”, whereby the guidewire is fed through the working channel of a flexible bronchoscope and through the target bronchial passageway. The bronchoscope is then removed from the bronchial tree while leaving the guidewire in place. This is an effective, but somewhat difficult procedure. The guidewire is typically quite long so that it can reach into the bronchial tree, which makes removal of the bronchoscope while keeping the guidewire in place quite difficult. The difficulty arises in that the guidewire can catch onto the inside of the working channel while the bronchoscope is being removed so that the bronchoscope ends up dislodging the guidewire tip from the target bronchial lumen or pulling the guidewire out of the bronchial tree. In view of this difficulty, it would be advantageous to develop an improved method and device for performing the guidewire exchange technique. It would also be advantageous to develop improved methods and devices for delivering bronchial isolation devices into the lung of a patient.

### SUMMARY

Disclosed are various devices and methods for delivering bronchial isolation devices into the lung of a patient. In accordance with one aspect of the invention, there is disclosed an apparatus for deploying a bronchial device in a bronchial passageway in a lung of a patient. The apparatus comprises a flexible shaft having an inner lumen and a distal end adapted to engage the bronchial isolation device; a wire slidably disposed in the inner lumen; and a housing movably coupled to the distal end of the shaft and configured to receive the bronchial device. The wire is connected to the housing to produce relative movement between the housing and the shaft to deploy the bronchial device from the housing.

Also disclosed is a guidewire for guiding a bronchial device into a bronchial passageway in a lung, the bronchial device having an inner lumen. The guidewire comprises an elongate flexible wire having a distal end configured for introduction through the patient's trachea into the bronchial passage. The wire is slidably positionable in the inner lumen of the bronchial device. The guidewire further comprises an anchor coupled to the wire, the anchor being configured to engage a wall of the bronchial passage to retain the position of the guidewire therein.

Also disclosed is a method of positioning a device in a bronchial passageway in a patient's lung. The method comprises positioning a guidewire in the bronchial passageway, the guidewire having an anchor coupled thereto; engaging the anchor to a wall of the bronchial passage to retain the position of the guidewire therein; inserting the guidewire through a lumen in the device; and sliding the device along the guidewire into the bronchial passage.

Also disclosed is a method of deploying a bronchial device in a bronchial passageway in a patient's lung. The method comprises positioning a bronchoscope in the patient's lung, the bronchoscope having a working channel; positioning a shaft of a delivery catheter through the working channel; coupling a housing to a distal end of the shaft while the shaft is positioned in the working channel; advancing the delivery catheter with the housing carrying the bronchial device until the housing is positioned in the bronchial passageway; and releasing the bronchial device from the housing.

Also disclosed is a method of deploying a bronchial device in a bronchial passageway in a patient's lung. The method comprises providing a bronchoscope, the bronchoscope having a working channel; positioning a shaft of a delivery catheter through the working channel, the shaft having a housing coupled to its distal end, the housing carrying the bronchial device; coupling a handle to a proximal end of the shaft with the shaft positioned in the working channel, the handle having a movable actuator; positioning the bronchoscope in the patient's lung with the shaft positioned in the working channel; advancing the delivery catheter until the housing is positioned in the bronchial passageway; and moving the actuator to release the bronchial device.

Also disclosed is a method of deploying a bronchial device in a bronchial passageway in a patient's lung. The method comprises positioning a bronchoscope in the patient's lung, the bronchoscope having a working channel; positioning a delivery catheter through the working channel, the delivery catheter having a housing carrying the bronchial device; advancing the delivery catheter until the housing is positioned in the bronchial passageway; locking the delivery catheter in position relative to the working channel; and releasing the bronchial device from the housing.

Other features and advantages of the present invention should be apparent from the following description of various embodiments, which illustrate, by way of example, the principles of the invention.

### BRIEF DESCRIPTION OF THE DRAWINGS

FIG. 1 shows an anterior view of a pair of human lungs and a bronchial tree with a bronchial isolation device implanted in a bronchial passageway to bronchially isolate a region of the lung.

FIG. 2 illustrates an anterior view of a pair of human lungs and a bronchial tree.

FIG. 3 illustrates a lateral view of the right lung.

FIG. 4 illustrates a lateral view of the left lung.

FIG. 5 illustrates an anterior view of the trachea and a portion of the bronchial tree.

FIG. 6 shows a perspective view of a bronchoscope.

FIG. 7 shows an enlarged view of a distal region of a bronchoscope.

FIG. 8 shows a delivery catheter for delivering a bronchial isolation device to a target location in a body passageway.

FIG. 9 shows a perspective view of a distal region of the delivery catheter.

FIG. 10 shows a plan, side view of the distal region of the delivery catheter.

FIG. 11 shows the delivery catheter housing containing a flow control device and implanted at a location L of a bronchial passageway.

FIG. 12 shows the delivery catheter deploying the flow control device at the location L of the bronchial passageway.

FIG. 13A shows a bronchoscope that utilizes a fixing mechanism to fix the position of the delivery catheter relative to the working channel of the bronchoscope.

FIG. 13B shows a cross-sectional view of a portion of the bronchoscope with the delivery catheter positioned in the working channel wherein the catheter includes an expandable member that engages the bronchoscope.

FIG. 13C shows a cross-sectional view of a portion of the bronchoscope with the delivery catheter positioned in the working channel wherein the bronchoscope includes an expandable member that engages the catheter.

FIG. 13D shows a cross-sectional view of a portion of the bronchoscope with the delivery catheter positioned in the working channel wherein a wedge member is positioned between the catheter and the wall of the working channel.

FIG. 14 shows a cross-sectional view of a distal region of a bronchoscope with a bronchial isolation device positioned directly within the working channel.

FIG. 15 shows the bronchoscope of FIG. 14 with a push wire having a handle protruding outward from the working channel entry port.

FIG. 16 shows a balloon-tipped guidewire located in a bronchial passageway, wherein a balloon is used to anchor the guidewire in a fixed position relative to the bronchial passageway.

FIG. 17 shows a frame-tipped guidewire located in a bronchial passageway, wherein an expanded frame is used to anchor the guidewire in a fixed position relative to the bronchial passageway.

FIG. 18A shows an exemplary guidewire having an anchor mounted on a distal end, the anchor shown in a collapsed state.

FIG. 18B shows an exemplary guidewire having an anchor mounted on a distal end, the anchor shown in an expanded state.

FIG. 19A shows a distal region of one embodiment of a guidewire including a schematic representation of the expandable anchor frame.

FIGS. 19B and 19C show an exemplary guidewire having an anchor frame that radially expands when retracted relative to the guidewire.

FIG. 20 shows an exemplary guidewire having an anchor frame that radially expands when extended relative to the guidewire.

FIG. 21A shows an unattached, removable control handle along with a delivery catheter positioned within a bronchoscope and a delivery housing protruding from the distal end of the bronchoscope.

FIG. 21B shows an enlarged view of a distal region of a bronchoscope with a proximal end of the delivery catheter being inserted into the distal end of the working channel.

FIG. 22 shows a delivery catheter with an attached, removable control handle positioned within a bronchoscope and a delivery housing protruding from the distal end of the bronchoscope.

FIG. 23 shows an enlarged view of the distal end of a bronchoscope with a housing positioned on a distal end of a catheter located in a working channel of the bronchoscope, wherein the housing has a diameter larger than the diameter of the working channel.

FIG. 24 shows an enlarged view of the distal end of a bronchoscope with a housing positioned on a distal end of a catheter located in a working channel of the bronchoscope, wherein a portion of the housing has a diameter larger than the diameter of the working channel.

FIG. 25 shows an enlarged view of the distal end of a bronchoscope with a housing positioned on a distal end of a catheter located in a working channel of the bronchoscope, wherein the housing is eccentrically-mounted.

FIG. 26 shows an enlarged view of the distal end of a bronchoscope with a housing positioned on a distal end of a catheter located in a working channel of the bronchoscope, wherein the housing has an eccentric shape.

FIG. 27 shows an enlarged view of the distal end of a bronchoscope with a housing positioned on a distal end of a catheter located in a working channel of the bronchoscope, wherein the housing is removably attachable to the catheter.

FIG. 28 shows a first embodiment of a housing retraction delivery catheter for delivering a bronchial isolation device.

FIG. 29 shows a cross-sectional view of the first embodiment of the housing retraction delivery catheter with the housing in a non-retracted state.

FIG. 30 shows a cross-sectional view of the first embodiment of the housing retraction delivery catheter with the housing in a retracted state FIG. 31 shows a housing and attached pull wire of the catheter of FIG. 28.

FIG. 32 shows a distal region of the catheter of FIG. 28 without the housing.

FIG. 33 shows a cross-sectional view of a distal region of a second embodiment of the housing retraction delivery catheter.

### DETAILED DESCRIPTION

Unless defined otherwise, all technical and scientific terms used herein have the same meaning as is commonly understood by one of skill in the art to which the invention(s) belong. It should be noted that the various devices and methods disclosed herein are not limited to the treatment of emphysema, and may be used for various other lung diseases.

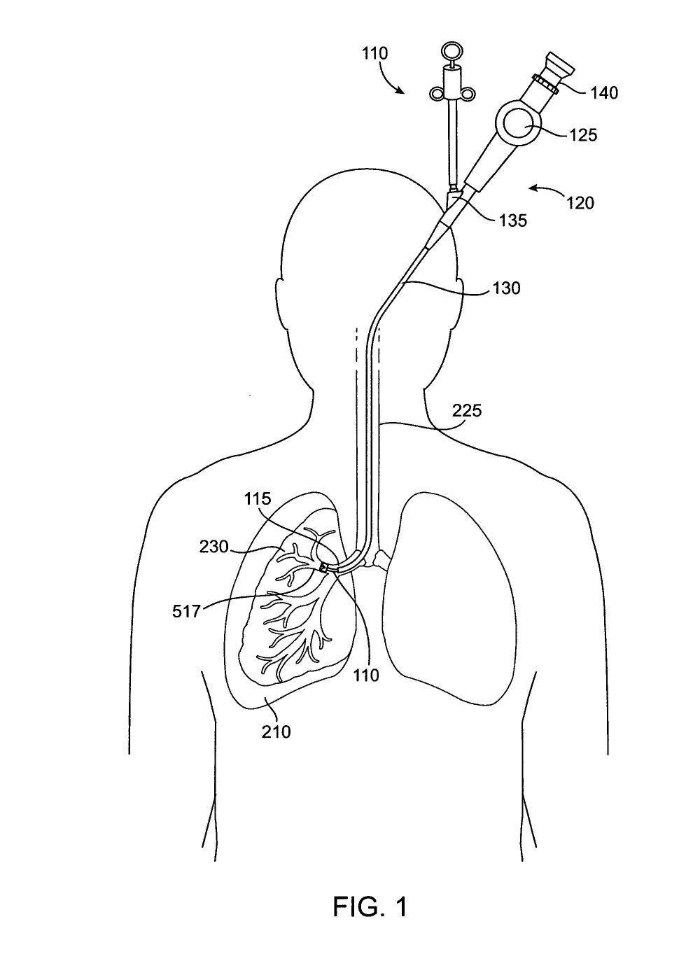

Disclosed are various devices and methods for delivering one or more bronchial isolation devices (which are sometimes referred to herein as flow control devices) to a location in a bronchial passageway. In several embodiments, the bronchial isolation device is delivered to the bronchial passageway by mounting the bronchial isolation device to the distal end of a delivery catheter and then inserting the delivery catheter into the bronchial passageway. In the example shown in FIG. 1, the delivery catheter 110 is inserted into the bronchial passageway so that the bronchial isolation device 115 is positioned at a desired location in the bronchial passageway 517. This can be accomplished by inserting the distal end of the delivery catheter 110 into the patient's mouth or nose, through the trachea, and down to the target location in the bronchial passageway 517. The delivery of the delivery catheter 110 to the bronchial passageway 517 can be accomplished in a variety of manners.

For example, the delivery catheter 115 can be deployed using a bronchoscope 120. As shown in FIG. 1, the delivery catheter 110 is inserted into the working channel of the bronchoscope 120, which is deployed to the bronchial passageway 517 either before or after the delivery catheter has been inserted into the bronchoscope 120. In an exemplary embodiment shown in FIG. 1, the bronchoscope 120 has a steering mechanism 125, a delivery shaft 130, a working channel entry port 135, and a visualization eyepiece 140. The bronchoscope 120 has been passed into a patient's trachea 125 and guided into the right primary bronchus 510 according to well-known methods.

The following references describe exemplary bronchial isolation devices and delivery devices: U.S. Pat. No. 5,954,766 entitled “Body Fluid Flow Control Device”; U.S. patent application Ser. No. 09/797,910, entitled “Methods and Devices for Use in Performing Pulmonary Procedures”; U.S. patent application Ser. No. 10/270,792, entitled “Bronchial Flow Control Devices and Methods of Use”; and U.S. patent application Ser. No. 10/448,154, entitled “Guidewire Delivery of Implantable Bronchial Isolation Devices in Accordance with Lung Treatment”. The foregoing references are all incorporated by reference in their entirety and are all assigned to Emphasys Medical, Inc., the assignee of the instant application.

### Exemplary Lung Regions

Throughout this disclosure, reference is made to the term “lung region”. As used herein, the term “lung region” refers to a defined division or portion of a lung. For purposes of example, lung regions are described herein with reference to human lungs, wherein some exemplary lung regions include lung lobes and lung segments. Thus, the term “lung region” as used herein can refer, for example, to a lung lobe or a lung segment. Such nomenclature conform to nomenclature for portions of the lungs that are known to those skilled in the art. However, it should be appreciated that the term “lung region” does not necessarily refer to a lung lobe or a lung segment, but can refer to some other defined division or portion of a human or non-human lung.

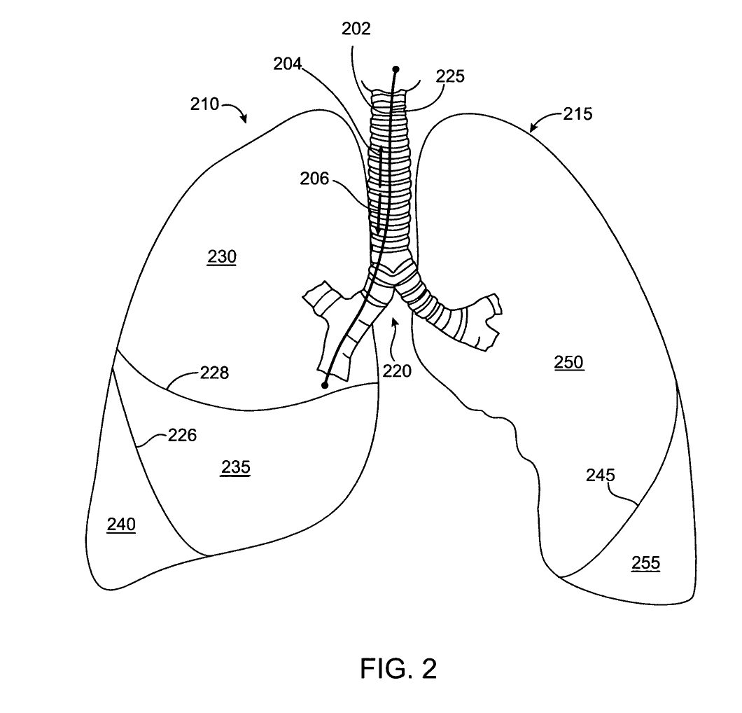

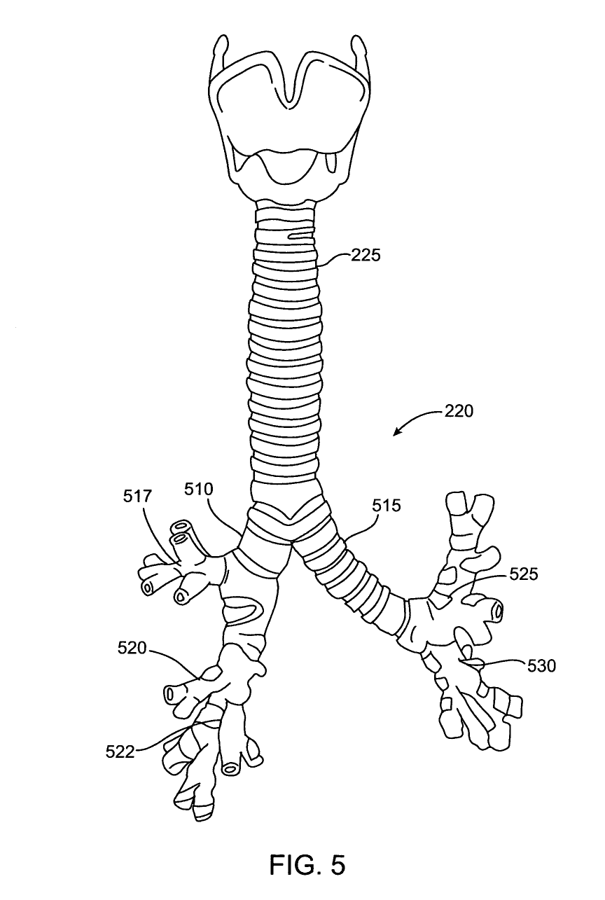

FIG. 2 shows an anterior view of a pair of human lungs 210, 215 and a bronchial tree 220 that provides a fluid pathway into and out of the lungs 210, 215 from a trachea 225, as will be known to those skilled in the art. As used herein, the term “fluid” can refer to a gas, a liquid, or a combination of gas(es) and liquid(s). For clarity of illustration, FIG. 2 shows only a portion of the bronchial tree 220, which is described in more detail below with reference to FIG. 5.

Throughout this description, certain terms are used that refer to relative directions or locations along a path defined from an entryway into the patient's body (e.g., the mouth or nose) to the patient's lungs. The path of airflow into the lungs generally begins at the patient's mouth or nose, travels through the trachea into one or more bronchial passageways, and terminates at some point in the patient's lungs. For example, FIG. 2 shows a path 202 that travels through the trachea 225 and through a bronchial passageway into a location in the right lung 210. The term “proximal direction” refers to the direction along such a path 202 that points toward the patient's mouth or nose and away from the patient's lungs. In other words, the proximal direction is generally the same as the expiration direction when the patient breathes. The arrow 204 in FIG. 2 points in the proximal or expiratory direction. The term “distal direction” refers to the direction along such a path 202 that points toward the patient's lung and away from the mouth or nose. The distal direction is generally the same as the inhalation or inspiratory direction when the patient breathes. The arrow 206 in FIG. 2 points in the distal or inhalation direction.

The lungs include a right lung 210 and a left lung 215. The right lung 210 includes lung regions comprised of three lobes, including a right upper lobe 230, a right middle lobe 235, and a right lower lobe 240. The lobes 230, 235, 240 are separated by two interlobar fissures, including a right oblique fissure 226 and a right transverse fissure 228. The right oblique fissure 226 separates the right lower lobe 240 from the right upper lobe 230 and from the right middle lobe 235. The right transverse fissure 228 separates the right upper lobe 230 from the right middle lobe 235.

As shown in FIG. 2, the left lung 215 includes lung regions comprised of two lobes, including the left upper lobe 250 and the left lower lobe 255. An interlobar fissure comprised of a left oblique fissure 245 of the left lung 215 separates the left upper lobe 250 from the left lower lobe 255. The lobes 230, 235, 240, 250, 255 are directly supplied air via respective lobar bronchi, as described in detail below.

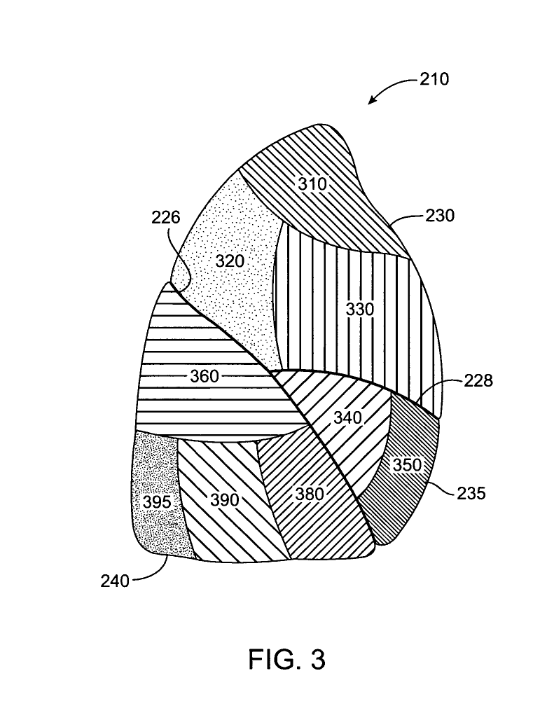

FIG. 3 is a lateral view of the right lung 210. The right lung 210 is subdivided into lung regions comprised of a plurality of bronchopulmonary segments. Each bronchopulmonary segment is directly supplied air by a corresponding segmental tertiary bronchus, as described below. The bronchopulmonary segments of the right lung 210 include a right apical segment 310, a right posterior segment 320, and a right anterior segment 330, all of which are disposed in the right upper lobe 230. The right lung bronchopulmonary segments further include a right lateral segment 340 and a right medial segment 350, which are disposed in the right middle lobe 235. The right lower lobe 240 includes bronchopulmonary segments comprised of a right superior segment 360, a right medial basal segment (which cannot be seen from the lateral view and is not shown in FIG. 3), a right anterior basal segment 380, a right lateral basal segment 390, and a right posterior basal segment 395.

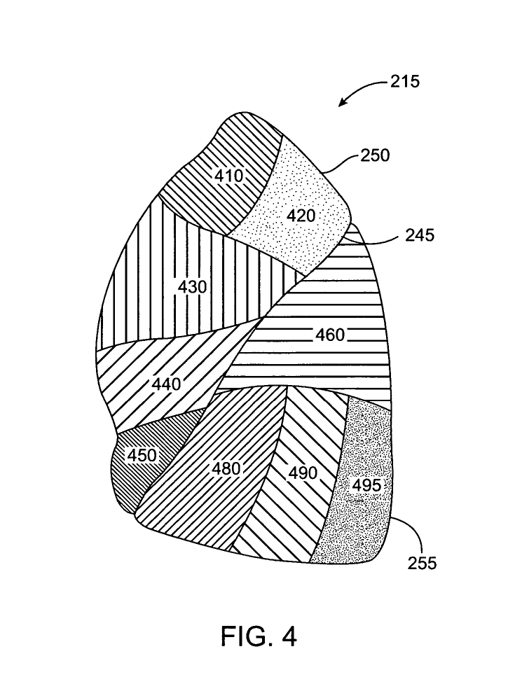

FIG. 4 shows a lateral view of the left lung 215, which is subdivided into lung regions comprised of a plurality of bronchopulmonary segments. The bronchopulmonary segments include a left apical segment 410, a left posterior segment 420, a left anterior segment 430, a left superior segment 440, and a left inferior segment 450, which are disposed in the left lung upper lobe 250. The lower lobe 255 of the left lung 215 includes bronchopulmonary segments comprised of a left superior segment 460, a left medial basal segment (which cannot be seen from the lateral view and is not shown in FIG. 4), a left anterior basal segment 480, a left lateral basal segment 490, and a left posterior basal segment 495.

FIG. 5 shows an anterior view of the trachea 325 and a portion of the bronchial tree 220, which includes a network of bronchial passageways, as described below. The trachea 225 divides at a lower end into two bronchial passageways comprised of primary bronchi, including a right primary bronchus 510 that provides direct air flow to the right lung 210, and a left primary bronchus 515 that provides direct air flow to the left lung 215. Each primary bronchus 510, 515 divides into a next generation of bronchial passageways comprised of a plurality of lobar bronchi. The right primary bronchus 510 divides into a right upper lobar bronchus 517, a right middle lobar bronchus 520, and a right lower lobar bronchus 422. The left primary bronchus 415 divides into a left upper lobar bronchus 525 and a left lower lobar bronchus 530. Each lobar bronchus 517, 520, 522, 525, 530 directly feeds fluid to a respective lung lobe, as indicated by the respective names of the lobar bronchi. The lobar bronchi each divide into yet another generation of bronchial passageways comprised of segmental bronchi, which provide air flow to the bronchopulmonary segments discussed above.

As is known to those skilled in the art, a bronchial passageway defines an internal lumen through which fluid can flow to and from a lung or lung region. The diameter of the internal lumen for a specific bronchial passageway can vary based on the bronchial passageway's location in the bronchial tree (such as whether the bronchial passageway is a lobar bronchus or a segmental bronchus) and can also vary from patient to patient. However, the internal diameter of a bronchial passageway is generally in the range of 3 millimeters (mm) to 10 mm, although the internal diameter of a bronchial passageway can be outside of this range. For example, a bronchial passageway can have an internal diameter of well below 1 mm at locations deep within the lung.

### Delivery System

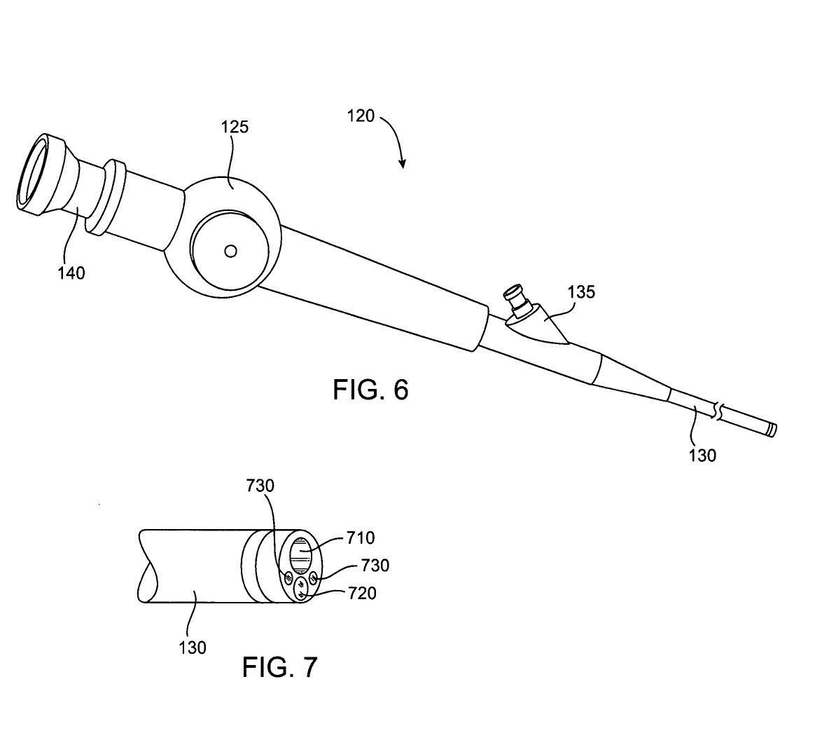

FIG. 6 shows an enlarged view of the bronchoscope 120, including the steering mechanism 125, delivery shaft 130, working channel entry port 135, and visualization eyepiece 140. In addition, the bronchoscope can also include a fiber optic bundle mounted inside the length of the bronchoscope for transferring an image from the distal end to the eyepiece 140. In one embodiment, the bronchoscope also includes a camera or charge-coupled device (CCD) for generating an image of the bronchial tree. FIG. 7 shows an enlarged view of the distal portion of the bronchoscope 120. A working channel 710 (sometimes referred to as a biopsy channel) extends through the delivery shaft 130 and communicates with the entry port 135 (shown in FIG. 6) at the proximal end of the bronchoscope 120. The working channel 710 can sometimes be formed by an extruded plastic tube inside the body of the bronchoscope 120. The bronchoscope 120 can also include various other channels, such as a visualization channel 720 that communicates with the eyepiece 140 and a pair of illumination channels 730.

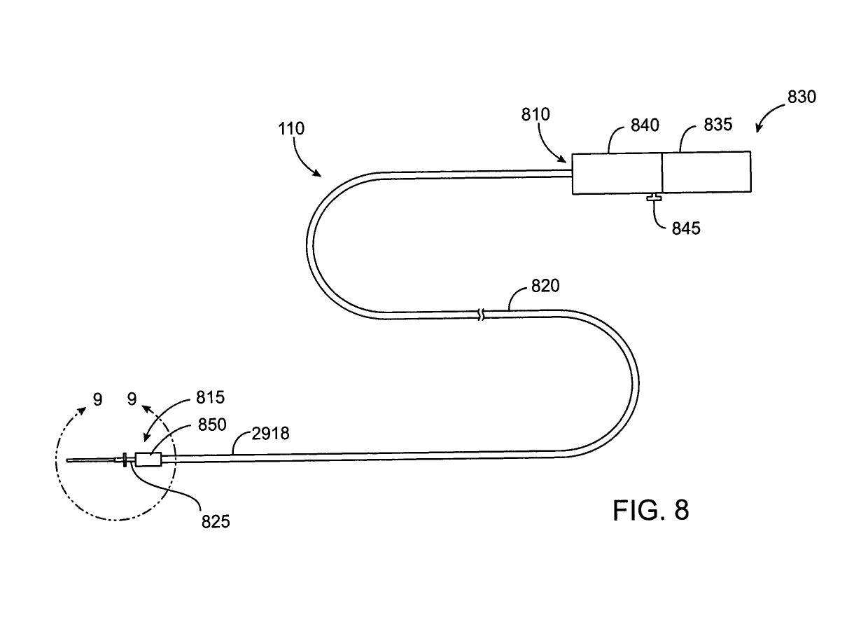

FIG. 8 shows one embodiment of the delivery catheter 110 for delivering and deploying the bronchial isolation device 115 to a target location in a bronchial passageway. The delivery catheter 110 has a proximal end 810 and a distal end 815 that can be deployed to a target location in a patient's bronchial passageway, such as through the trachea. The catheter 110 has an elongated outer member 820 and an elongated inner member 825 that is slidably positioned within the outer member 820 such that the inner member 825 can slidably move relative to the outer member 820 along the length of the catheter 130.

The following references describe exemplary delivery devices: U.S. Pat. No. 5,954,766 entitled “Body Fluid Flow Control Device”; U.S. patent application Ser. No. 09/797,910, entitled “Methods and Devices for Use in Performing Pulmonary Procedures”; U.S. patent application Ser. No. 10/270,792, entitled “Bronchial Flow Control Devices and Methods of Use”; and U.S. patent application Ser. No. 10/448,154, entitled “Guidewire Delivery of Implantable Bronchial Isolation Devices in Accordance with Lung Treatment”. The foregoing references are all incorporated by reference in their entirety and are all assigned to Emphasys Medical, Inc., the assignee of the instant application.

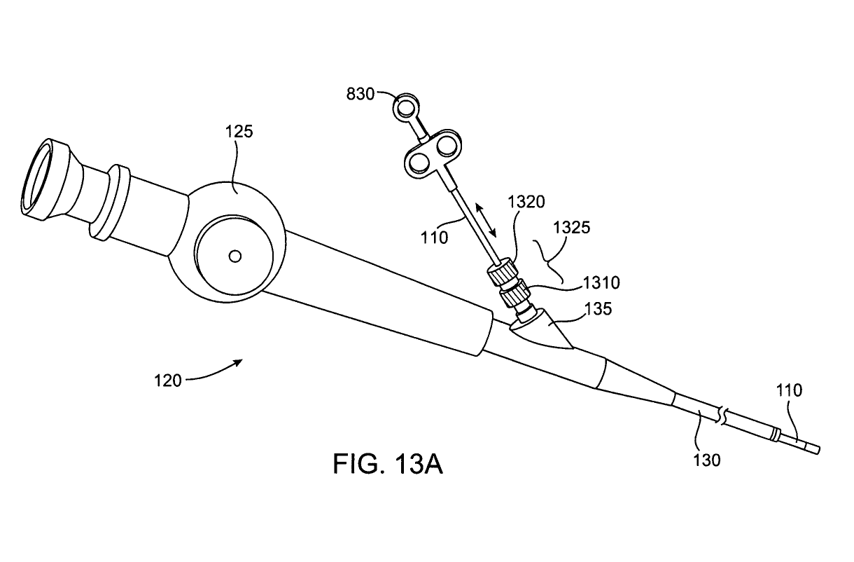

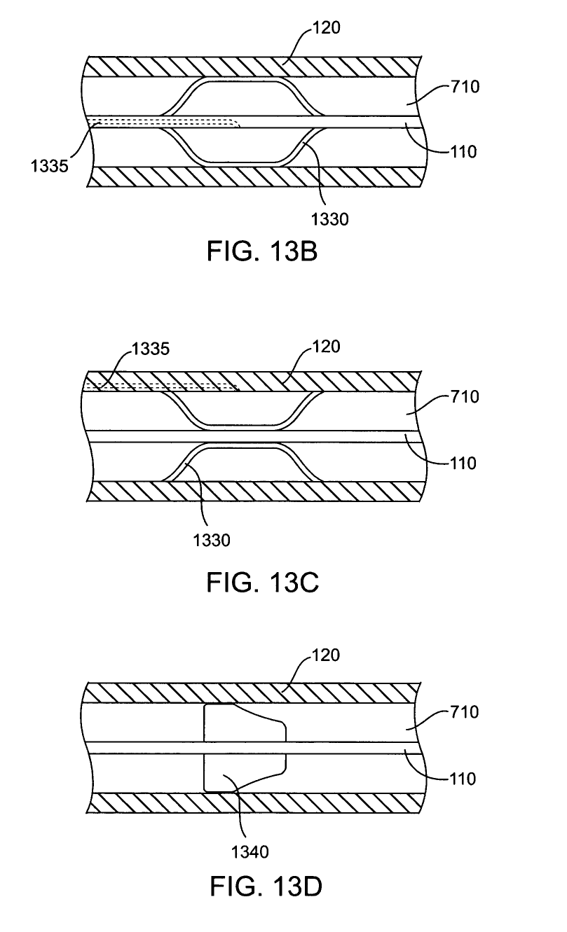

In this regard, an actuation member, such as a control handle 830, is located at the proximal end 810 of the catheter 110. The handle 830 can be actuated to move the inner member 825 relative to the outer member 820 (and vice-versa). In the illustrated embodiment, the handle 830 includes a first piece 835 and a second piece 840, which is slidably moveable relative to the first piece 835. The inner member 825 of the catheter 110 can be moved relative to the outer member 820 by slidably moving the first piece 835 of the handle 830 relative to the second piece 840. This can be accomplished, for example, by attaching the proximal end of the catheter inner member 825 to the first piece 835 of the handle 830 and attaching the proximal end of the catheter outer member 820 to the second piece 840. The actuation member could also take on other structural forms that use other motions to move the inner member 825 relative to the outer member 820. For example, the actuation member could have scissor-like handles or could require a twisting motion to move the inner member 825 relative to the outer member 820. FIG. 13A, described below, shows another exemplary embodiment of the handle 830 that includes holes in which the fingers of an operator can be inserted.

With reference to FIG. 8, the handle 830 can also include a locking mechanism 845 for locking the position of the first piece 835 relative to the second piece 840 to thereby lock the position of the inner member 825 of the catheter 110 relative to the outer member 820. The locking mechanism 845 can comprise, for example, a set screw or other suitable locking mechanism that can be used to lock the position of the first piece 835 of the handle 830 relative to the second piece 840.

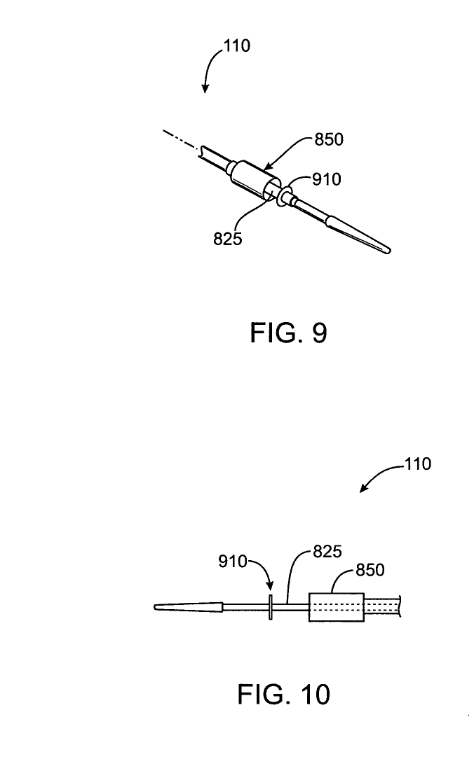

With reference still to FIG. 8, a housing 850 is located at or near a distal end of the catheter 110. In one embodiment, the housing 850 is attached to a distal end of the outer member 820 of the catheter 110 but not attached to the inner member 825. The housing 850 defines an inner cavity that is sized to receive the bronchial isolation device 115 therein. FIG. 9 shows an enlarged, perspective view of the distal portion of the catheter 110 where the housing 850 is located. FIG. 10 shows a plan, side view of the distal portion of the catheter 110 where the housing 850 is located. As shown in FIGS. 9 and 10, the housing 850 is shaped to receive the bronchial isolation device therein and is open at a distal end and closed at a proximal end. The inner member 825 of the catheter 110 protrudes through the housing 850 and can be slidably moved relative to the housing 850. An ejection member, such as a flange 910, is located at a distal end of the inner member 825. The ejection member can be used to eject the bronchial isolation device 115 from the housing 850. The flange 910 is sized such that it can be received into the housing 850. The housing can be manufactured of a rigid material, such as steel. The housing 850 can also be flexible or collapsible. Although the housing 850 is shown having a cylindrical shape, it should be appreciated that the housing 850 can have other shapes that are configured to receive the bronchial isolation device therein.

The inner member 825 of the catheter 110 can include a central guidewire lumen (not shown) that extends through the entire length of the catheter 110. The central guidewire lumen of the inner member 825 is sized to receive a guidewire, which can be used during deployment of the catheter 110 to guide the catheter 110 to a location in a bronchial passageway.

In use, the bronchial isolation device 115 is inserted into the housing 850 and the delivery catheter inserted into a bronchial passageway via the trachea such that the housing is located at or near a desired location in the bronchial passageway. The bronchial isolation device 115 is then ejected from the housing 850 into the bronchial passageway. In one embodiment, the bronchial isolation device 115 can transition between a compressed state and an expanded state. In the compressed state, the bronchial isolation device 115 has a reduced diameter that permits the device to fit within the housing 850 and be inserted through the trachea and into the bronchial tree. In the expanded state, the bronchial isolation device 115 has an increased diameter such that the device can anchor within the bronchial passageway. The bronchial isolation device 115 can be configured to automatically transition from the compressed state to the expanded state when the device is ejected from the housing 850.

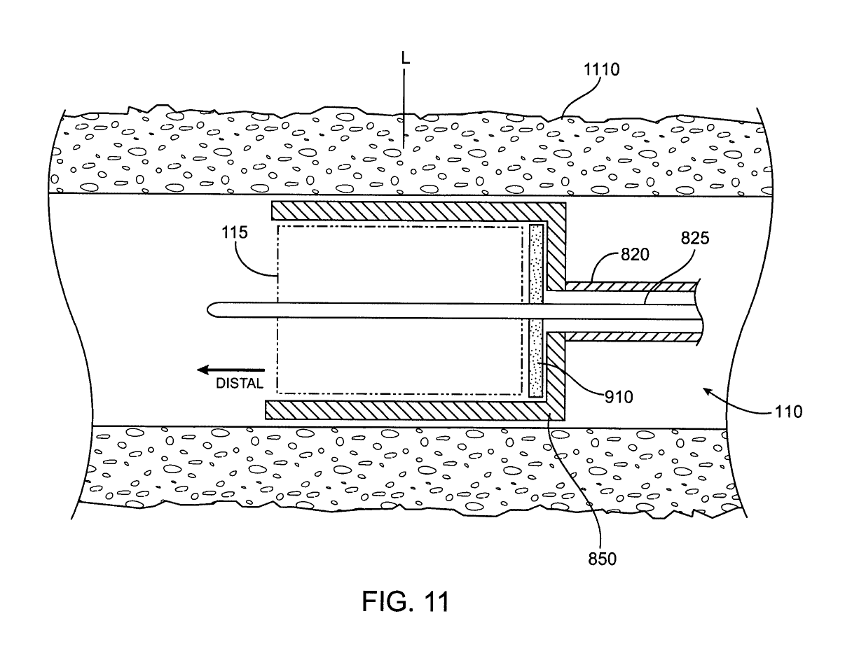

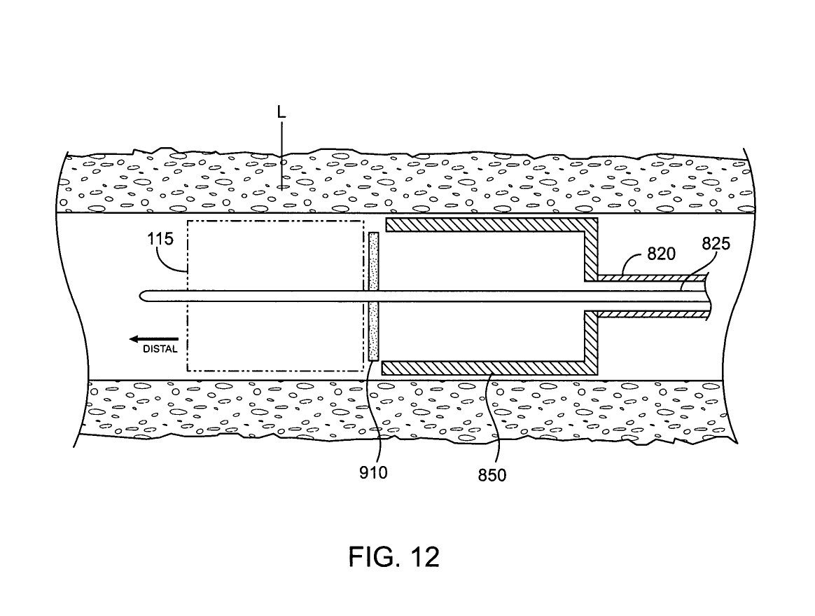

The ejection of the bronchial isolation device 115 from the housing 850 can be accomplished in a variety of ways. For example, as shown in FIG. 11, the catheter 110 is deployed to a target location L of a bronchial passageway 1110 such that the distal end of the catheter, including the housing 850, is located at or near the target location L. The catheter handle 830 is then actuated to move the outer catheter member 820 in a proximal direction relative to the location L, while maintaining the location of the bronchial isolation device 115, inner member 825, and flange 910 fixed with respect to the location L. The proximal movement of the outer member 820 causes the attached housing 850 to also move in a proximal direction, while the flange 910 prevents the bronchial isolation device 115 from moving in the proximal direction. This results in the housing 850 sliding away from engagement with the bronchial isolation device 115 so that the bronchial isolation device 115 is eventually entirely released from the housing 850 and implanted in the bronchial passageway at the target location L, as shown in FIG. 12. An anchor member can be location on the housing 850 for anchoring the housing 850 in place during deployment. The housing anchor member can comprise, for example, an expandable structure, such as an inflatable balloon, located on the periphery of the housing 850 for anchoring to the bronchial wall.

It should be appreciated that when the bronchial isolation device 115 needs to be placed into bronchial target sites located in the apical regions (either right or left) of the lungs, the bronchoscope 120 may have to bend through an acute angle in order to reach the most apical placement sites. For example, as shown in FIG. 1, the distal region of the bronchoscope 120 is bending through an angle of approximately 100 degrees. In certain circumstances, however, the bronchoscope may be required to bend as much as 180 degrees in order to reach target locations in the apex of the lung. When this happens, the working channel 710 of the bronchoscope must also bend acutely. In such cases, the effective diameter of the working channel 710 reduces in the region of the bend due to mild kinking or flattening of the working channel 710. Given that it can be desirable to fit as large a bronchial isolation device through the working channel 710 as possible in order to be able to treat larger bronchial lumens, this reduction in effective diameter of the working channel 710 can be a disadvantage as it would reduce the effective range of bronchial isolation devices that can be used.

Moreover, such bends in the distal region of the bronchoscope 120 can make it particularly difficult to advance the delivery catheter 110 through the bends in the working channel 710. One reason is that the distal region of the catheter 110 with the housing 850 is typically stiffer and more resistant to bending than the rest of the catheter 110. This makes it especially difficult to advance the stiffer regions of the delivery catheter through a bronchoscope working channel that is curved or bent.

One solution to this problem is to insert the delivery catheter 110 into the bronchoscope working channel 710 while the bronchoscope 120 is straight and the distal tip is not bent. The delivery catheter 110 can be inserted into the working channel 710 such that the distal end of the delivery catheter 110 is flush with the distal end of the bronchoscope. In such a case, the delivery catheter 110 is inserted into the bronchoscope 120 prior to inserting the bronchoscope 120 into the patient. This would allow the largest possible diameter delivery catheter to be inserted into the working channel 710, as the working channel is at its largest diameter when unbent.

After insertion of the delivery catheter 110 into the bronchoscope 120, the bronchoscope 120 is inserted into the trachea and bronchial tree of the patient, the distal tip is flexed as needed, and the scope is positioned just proximal to the target delivery site. Alternately, the catheter 110 can be inserted into the working channel 710 when the bronchoscope 120 is already located in the trachea of the patient but when the tip of the bronchoscope 120 is still straight and has not been bent. As before, the distal tip can then be flexed as needed, and the scope positioned just proximal to the target delivery site. The housing 850 on the distal end of the delivery catheter 110 is then advanced a small distance from the distal tip of the bronchoscope to the target implant site for deployment of the bronchial isolation device. Bronchoscopes typically have a short section at the distal tip that does not flex when the scope is steered, and if the more rigid section containing the housing 850 and compressed isolation device 850 is located in this portion of the bronchoscope 120, the steering capacity of the bronchoscope should not be dramatically impeded.

Alternately, in cases where it is difficult to advance the delivery catheter 110 through the bronchoscope 120 once the distal tip of the bronchoscope 120 is bent, the delivery catheter 110 can be inserted into the working channel 710 so that the distal end of the delivery catheter 110 extends past the distal end of the bronchoscope 120. The portion of the delivery catheter 110 containing the compressed isolation device 115 is positioned just distal to the distal end of the bronchoscope 120. This way, the more rigid portion of the delivery catheter 110 is not inside the working channel 710, and the delivery catheter 110 either does not need to be distally advanced or needs to be advanced minimally relative to the bronchoscope 120 in order to deploy the isolation device 115 in the target bronchial lumen.

In either of these delivery methods, it may be advantageous or even required to fix the position of the delivery catheter 110 relative to the working channel 710 at various times during device delivery. A device for fixing the position of the delivery catheter 110 relative to the working channel 710 of the bronchoscope 120 is now described. With reference to FIG. 13A, the bronchoscope 120 has a connector 1310, such as a Luer Lock connector, mounted to the working channel entry port 135. A second connector 1320, such as a Touhy-Borst connector, is located on the delivery catheter 110 and can lock onto the connector 1310 at the working channel entry port 135. The second connector 1320 has a center channel sized to receive the delivery catheter 110 therethrough. In addition, the second connector 1320 can lock onto to the delivery catheter 110 at any position along the length of the delivery catheter 110. In one embodiment, the second connector 1320 locks onto the delivery catheter 110 by compression against the delivery catheter 110 in a tight, frictional fit.

The second connector 1320 may be removably attached to the connector 1310. In use, the delivery catheter 110 is inserted into the working channel 710 and the delivery catheter 110 is positioned relative to the working channel 710 in a desired manner, such as with the distal end of the delivery catheter 110 positioned flush with the distal end of the bronchoscope 120. The second connector 1320 is then locked to the first connector 1310 and also locked to the delivery catheter 110, thereby fixing the position of the delivery catheter 110 relative to the bronchoscope 120 and working channel 710. In this way, the position of the delivery catheter 110 relative to the bronchoscope 120 and working channel 710 can be fixed, such as with the distal end of the delivery catheter 110 either flush with the distal end of the bronchoscope 120, or with the distal end of the delivery catheter positioned just past the distal end of the bronchoscope 120. This way, the operator would not have to hold both the bronchoscope 120 and the delivery catheter 110 while the bronchoscope 120 is inserted into the bronchial tree. Alternatively, connectors 1310 and 1320 may be combined into a single connector assembly 1325. The connector assembly 1325 is attached to and removed from the bronchoscope 120 by attaching it to the entry port 135 using a luer connection or through other means.

FIG. 13B shows a cross-sectional view of a portion of the bronchoscope 120 with the delivery catheter 110 position in the working channel 710. In the alternative embodiment shown in FIG. 13B, an expandable member, such as an inflatable balloon 1330, is positioned on the shaft of the catheter 110 for frictionally engaging the wall of the working channel 710. The expand balloon 1330 can be inflated via an inflation lumen 1335 so that the balloon expands and frictionally engages the wall of the working channel 710 and thereby secure the position of the catheter 110 relative to the bronchoscope 120. In another embodiment, shown in FIG. 13C, the expandable member comprised of a balloon 1330 is attached to the bronchoscope 120 and located on the wall of the working channel 710. The balloon 1330 can be inflated via an inflation lumen 1335 in the bronchoscope 120 so that so that the balloon 1330 expands and frictionally engages the catheter 110 to secure the position of the catheter 110 relative to the bronchoscope 120. It should be appreciated that the expandable member can comprise any other suitable expandable structure, such as an expandable frame.

In another embodiment, shown in FIG. 13D, the expandable member is replaced by a wedge member 1340 that is positioned between the catheter 110 and the wall of the working channel 710. The wedge member 1340 can be slidably mounted over the catheter 110 and can be wedged between the catheter 110 and the wall of the working channel 710 to frictionally engage the catheter 110 and the bronchoscope 120. The frictional engagement serves to secure the position of the catheter 110 relative to the bronchoscope 120. When it is desired to move the catheter 110 relative to the bronchoscope 120, the wedge member 1340 can be removed from frictional engagement, thereby freeing the catheter 110 for movement. The wedge member 1340 can be positioned in the working channel 710 near the working channel entry port 135 (shown in FIG. 13A) to facilitate access to the wedge member 1340.

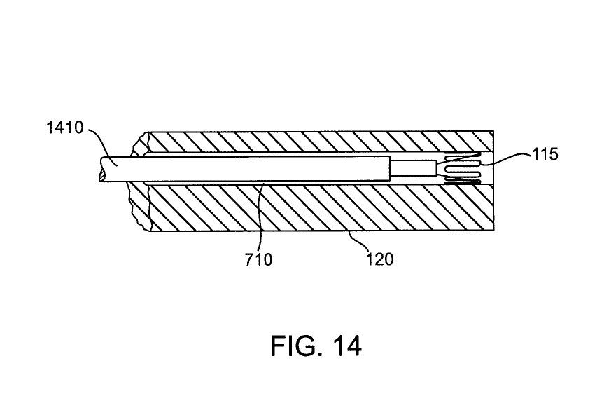

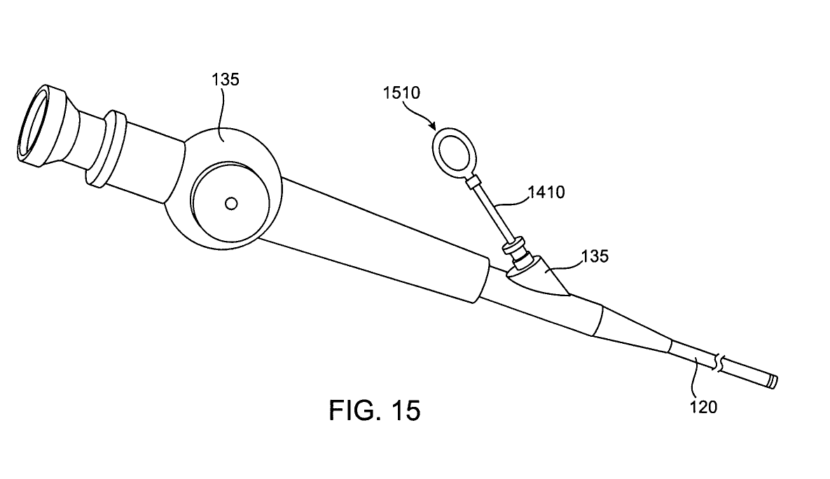

It should be appreciated that the use of the delivery catheter 110 can be eliminated such that the bronchial isolation device 115 is compressed and inserted directly into bronchoscope working channel 710, such as shown in the cross-sectional view of the bronchoscope distal region of FIG. 14. During delivery of the bronchial isolation device 115, the bronchoscope is inserted into the patient's bronchial tree so that the distal end of the bronchoscope 120 is located at or near the implant site, such as just proximally to the target implant site. An elongate, flexible pushing device, such as a catheter, rod, or wire 141, is inserted into the working channel entry port 135 of the working channel 710, run down the length of the working channel 710, and used to push the bronchial isolation device 115 out of the bronchoscope 120 through the opening at the distal end of the working channel 710. In this manner, the bronchial isolation device 115 can be inserted into the target bronchial lumen. FIG. 15 shows the bronchoscope 120 with the push wire 1410 having a handle 1510 protruding outward from the working channel entry port 135. The push wire 1410 can be manipulated by moving the handle 1510.

### Anchorable Guidewire Assisted Delivery System

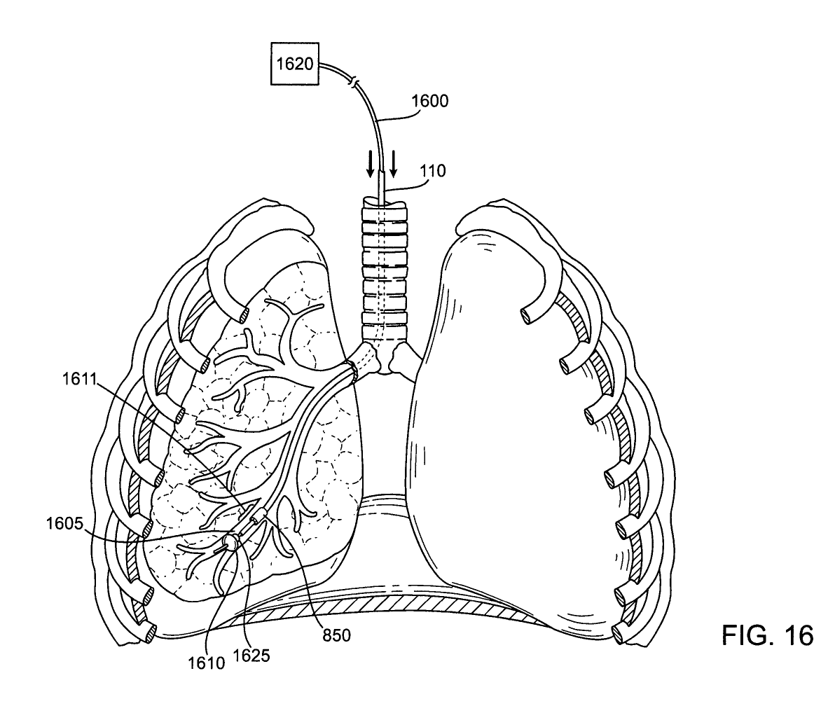

FIG. 16 shows a delivery system that can be used to deliver a guidewire 1600 to a location in a bronchial passageway according to an exchange technique. As previously discussed, according to the exchange technique, the guidewire 1600 is fed through the working channel of a bronchoscope (not shown in FIG. 16) and to the target bronchial passageway 1605. The bronchoscope is then removed from the bronchial tree while leaving the guidewire 1600 in place. In order to prevent the guidewire 1600 from being displaced or dragged out of the bronchial passageway 1605 during removal of the bronchoscope, a distal end of the guidewire 1600 is fixed to the interior surface of the bronchial passageway 1605 to anchor the guidewire 1600 in place. Alternatively, the guidewire 1600 may be placed into a location in a bronchial passageway using any other method. One such method involves temporarily attaching the guidewire 1600 to the outside of the bronchoscope using devices and methods described in U.S. patent application Ser. No. 10/448,154, entitled “Guidewire Delivery of Implantable Bronchial Isolation Devices in Accordance with Lung Treatment”, which is incorporated by reference in its entirety and is assigned to Emphasys Medical, Inc., the assignee of the instant application

Once the guidewire 1610 has been positioned, the delivery catheter 110 with a bronchial isolation device contained in the housing 850 is threaded over the guidewire 1610 and advanced along the guidewire 1610 until the housing 850 is located in the target location 1611 in the lungs. The target location 1611 can be located in a part of the lung, such as the apical lobes, that requires the delivery catheter 110 to bend through an acute angle in order to reach the target bronchial passageway. In such a case, there is an increased risk that the guidewire 1600 will undesirably move within the bronchial passageway if the catheter pulls or pushes the guidewire out of the bronchial passageway. Advantageously, the fixation of the distal end of the guidewire 1600 to the interior surface of the bronchial wall reduces the likelihood of the delivery catheter moving the guidewire 1600 during advancement of the delivery catheter 110 over the guidewire 1600.

The guidewire 1600 can be fixed to various locations in the bronchial passageway, such as at a location distal to the target site. There are various ways to temporarily anchor the distal end of the guidewire 1600 to the bronchial wall. For example, the distal end of the guidewire 1600 can have an anchor device that temporarily attaches to the bronchial wall. In one embodiment, the anchor device comprises an inflatable balloon. As shown in FIG. 16, an inflatable balloon 1610 is positioned at the distal end of the guidewire 1600, which includes an inflation lumen (not shown) that extends through the guidewire from end-to-end. An inflation device 1620, such as a pump or syringe, is removably coupled to the proximal end of the guidewire 1600 for inflating the balloon 1610 through the inflation lumen in the guidewire 1600. The inflation device 1620 can be removably attached to the proximal end of the guidewire 1600 to allow the delivery catheter 110 to be inserted over the proximal end of the guidewire 1600 and advanced down the guidewire 1600 to the target bronchial site 1611. In one embodiment, the guidewire 1600 has a small one-way valve 1625 located between the inflation device 1620 and the balloon 1610. The one-way valve 1625 can be closed to prevent air from escaping from the balloon 1610.

In use, the guidewire 1600 is positioned in the bronchial tree such that the distal end and the balloon 1610 are located just distal to the target site 1611. The balloon 1610 is then inflated using the inflation device 1620. When the balloon 1610 inflates, it radially expands to a size such that the balloon presses against the bronchial walls and grips the walls at the target site 1611. The balloon 1610 exerts sufficient pressure against the bronchial walls to retain the balloon 1610 in place and thereby secure the distal end of the guidewire 1600 relative to the bronchial wall. Once the distal end of the guidewire 1600 is secured to the bronchial wall, the inflation device 1620 can be removed from the guidewire 1600 and the bronchoscope, if present, is also removed. The delivery catheter 110 is then advanced down the guidewire 1600 to the target site of the bronchial passageway. When it is desired to remove the guidewire 1600, the one-way valve 1625 is opened to permit air to escape from the balloon 1610. The balloon 1610 deflates, reduces in size, and disengages from the bronchial wall. The guidewire 1600 can then be either repositioned or removed from the patient.

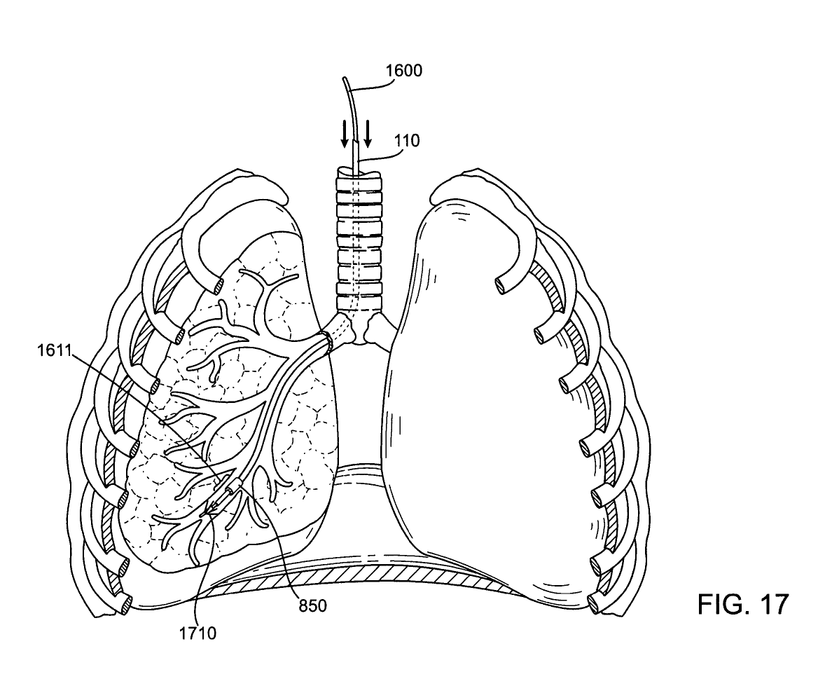

In an alternate embodiment, shown in FIG. 17, the anchor device on the guidewire 1600 comprises an expandable frame 1710 located at the distal end of the guidewire 1600. The frame 1710 can include means of securing itself to the bronchial wall, such as spikes or prongs, to prevent the guidewire 1600 from moving in the proximal direction when positioned in the bronchial passageway. The guidewire 1600 includes a mechanism on the proximal end for expanding and retracting the expandable frame 1700, either in the form of a removable actuation handle, or through some other mechanism.

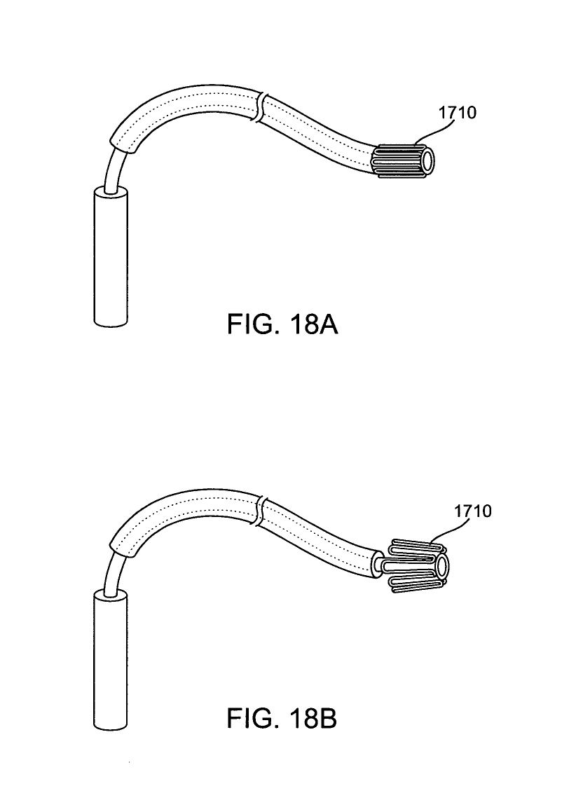

FIG. 18A shows an exemplary guidewire having an expandable frame 1710 mounted on a distal end. The frame 1710 can expand from a first, contracted state to a second, expanded state that can anchor within a bronchial lumen. In the contracted state, shown in FIG. 18A, the frame 1710 is small enough to slide freely through the lumen of the bronchial passageway. FIG. 18B shows the frame in an expanded state, wherein the diameter of the frame 1710 has increased to a size that will engage the walls of the bronchial passageway. The frame 1710 can have one or more prongs that anchor into the walls of the bronchial passageway.

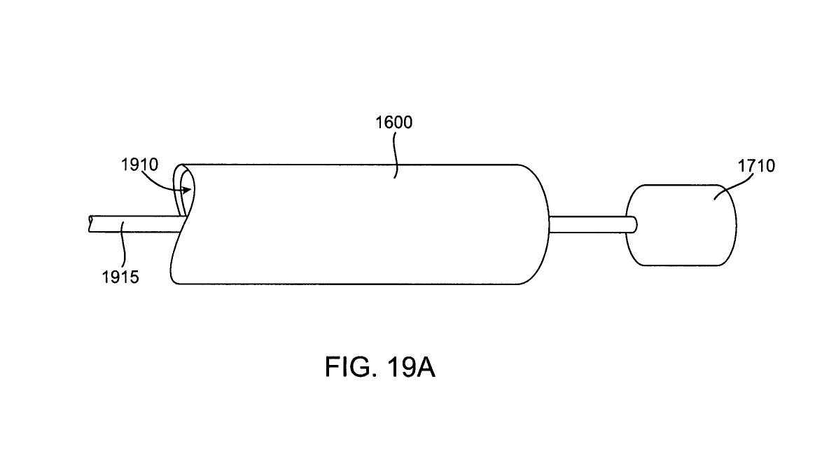

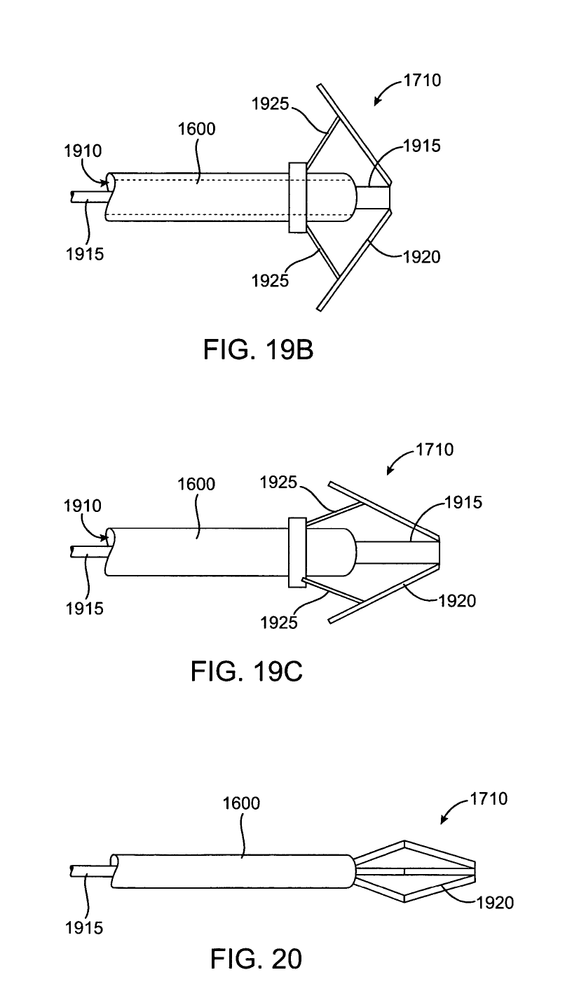

FIG. 19A shows a distal region of one embodiment of the guidewire 1600 including a schematic representation of the expandable frame 1710. An oval is used in FIG. 19A to represent the frame 1710, although it should be appreciated that the frame can have various structures, some of which are described below. An internal lumen 1910 extends through the guidewire 1600 and an inner wire 1915 is slidably positioned in the internal lumen 1910. The frame 1710 is attached to the distal end of the inner wire 1915. An operator can slide the inner wire 1915 relative to the guidewire 1600, such as by manipulating an actuator located at the distal end of the guidewire 1600. Thus, the frame 1710 can be proximally retracted into the guidewire 1600 by sliding the inner wire 1915 in the proximal direction.

In one embodiment, the frame 1710 is configured to radially expand in size when the inner wire 1915 is retracted and to radially collapse in size when the inner wire 1710 is extended relative to the guidewire 1600. For example, with reference to FIG. 19B and 19C, in one embodiment the frame 1710 includes one or more struts 1920 that extend radially outward from the inner wire 1915. The struts are attached at a distal end to the inner wire 1915 and are slidably attached to links 1925 that are pivotably attached to a ring 1930 fixed to the outer guidewire 1600. When the inner wire 1915 is retracted, the struts 1920 slide relative to the links 1925 and expand radially outward, as shown in FIG. 19B. When the inner wire 1915 is extended relative to the guidewire 1600, the struts 1920 slide relative to the links 1925 and contract radially inward, as shown in FIG. 19C.

FIG. 20 shows another embodiment wherein the anchor frame 1710 radially contracts in size when the inner wire 1915 is retracted. The frame 1710 radially expands in size when the inner wire 1710 is extended relative to the guidewire 1600. In this embodiment, the frame 1710 comprises outwardly-biased struts 1920 that collectively form a ring that is biased to expand radially outward. When the inner wire 1915 is retracted, the frame 1710 is drawn into the lumen 1910 in the guidewire 1600 so that the guidewire 1600 radially constricts the frame 1710. When the inner wire is extended relative to the guidewire 1600, the frame 1710 is gradually released from constriction such that the outward bias in the frame causes it to radially expand in size. It should be appreciated that other structures and mechanisms can be used to expand and contract the frame 1710.

### Muzzle Loaded Delivery System

As discussed, the bronchial isolation device 115 can be delivered using the catheter 110, which has the housing 850 located at or near a distal end of the catheter 110. The housing is configured to receive the bronchial isolation device 115 therein. A bronchoscope can be used to deliver the delivery catheter 110 to the bronchial passageway by inserting the delivery catheter 110 into the working channel 710 via the working channel entry port 135 (shown in FIG. 6).

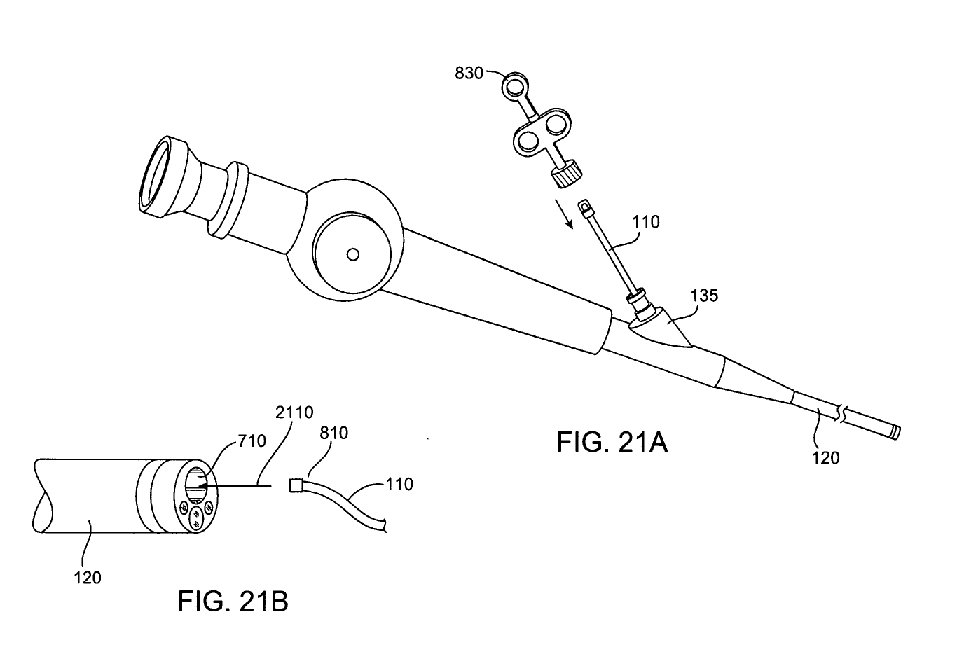

In certain circumstances, the size of the housing 850 may be too large to fit within the working channel 710 or through the working channel entry port 135. In such circumstances, the proximal end of the delivery catheter should be inserted into the working channel 710 through the distal end of the bronchoscope 120, which avoids having to insert the housing 850 through the working channel. However, the handle 830 on the distal end of the delivery catheter may not be small enough to fit in the working channel so that the delivery catheter cannot be inserted. FIG. 21A show an embodiment of the delivery catheter 110 that overcomes this problem. The handle 830 is removably mounted at or near the proximal end 810 of the delivery catheter 110. The handle 830 can be removably mounted using various means, such as a male-female configuration that mates in a press-fit manner or in a threaded relationship.

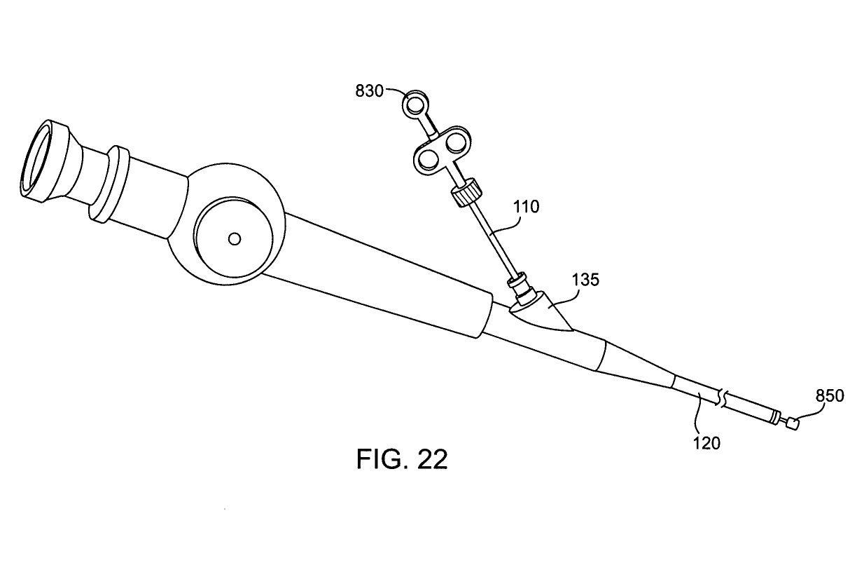

As shown in FIG. 21B, when the handle 830 is removed from the delivery catheter 110, the proximal end 810 of the catheter 110 has a diameter that is sufficiently small to fit within the working channel 710 of the bronchoscope 120. The proximal end 810 of the delivery catheter 110 is then inserted into the distal end of the working channel 710 (as exhibited by the arrow 2110) and slid therethrough until the proximal end 810 protrudes outward through the working channel entry port 135 and the housing 850 protrudes out of the distal end of the bronchoscope 120, as shown in FIG. 22. The handle 830 may then be reattached to the delivery catheter 110 to allow delivery of the bronchial isolation device.

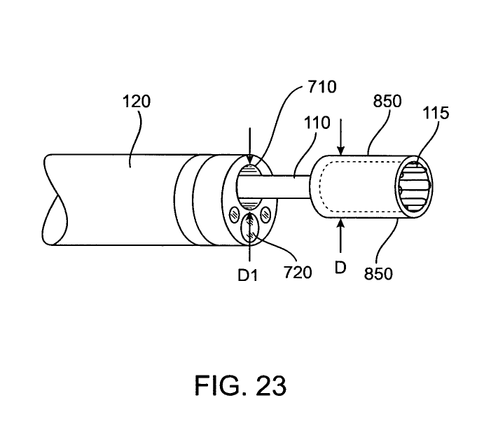

FIG. 23 shows an enlarged view of the distal region of the bronchoscope 120 with the delivery housing 850 protruding out of the distal end of the bronchoscope 120. In this embodiment, the housing 850 has a transverse dimension D (such as the diameter for a cylindrical housing) that is larger than the transverse dimension D1 of the working channel 710. Thus, the housing 850 is too large to fit within the working channel 710. Such a housing 850 can be used where the size of the entire compressed bronchial isolation device 115 is larger than the working channel 710 of the bronchoscope, or where just a portion of the compressed bronchial isolation device is larger than the working channel 710.

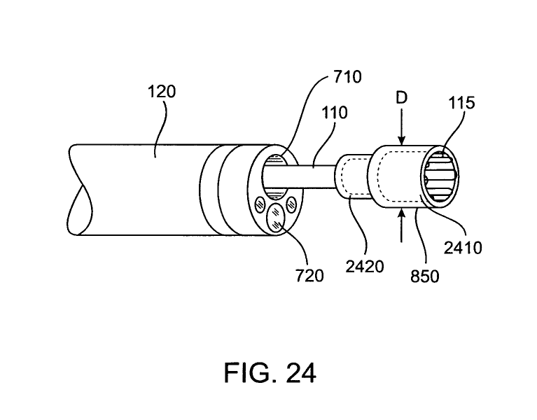

In another embodiment, shown in FIG. 24, the housing 850 is multi-sized. The housing has a first portion 2410 that has a transverse dimension larger than that of the working channel 710, and a second portion 2420 that has transverse dimension smaller than that of the working channel 710. Thus, the second portion 2410 is sized to fit within the working channel 710 while the first portion 2410 does not fit within the working channel 710. The first portion 2410 of the delivery catheter can contain the entire compressed bronchial isolation device 115. Alternately, if only part of the bronchial isolation device 115 cannot be compressed to a diameter that will fit in the working channel 710, then the first portion 2410 and the second portion 2420 can both contain a portion of the bronchial isolation device 115. During deployment of the delivery catheter using the bronchoscope, the delivery catheter 110 is positioned within the working channel 710 so that the second portion 2420 is within the working channel 710 and the first portion 2420 protrudes out of the working channel 710. In one embodiment, the distal end of the delivery catheter (i.e., the second portion 2420 of the housing 850) extends as little as possible beyond the distal end of the bronchoscope 120. The second portion 2420 may have a diameter selected to slide easily within the working channel 710, or to fit frictionally therein to maintain it in place. Alternatively, a snap detent, threaded surface, or other connection may be provided on the second portion 2420 to provide a positive engagement in the working channel 710.

In FIG. 24, the housing 850 is shown having a “stepped” size configuration, such that the diameter of the housing 850 undergoes a sudden decrease moving from the first portion 2410 to the second portion 2420. The housing 850 in FIG. 24 has two portions of different size. It should be appreciated that the variation in size can also be gradual (e.g., sloped or conical) and that the housing 850 can have more than two sections of varying size.

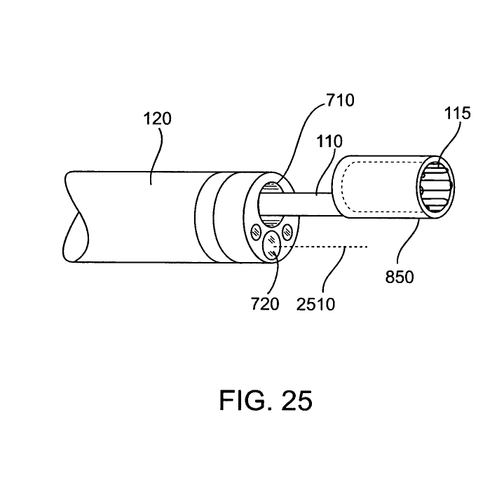

As discussed above with reference to FIG. 7, the bronchoscope 120 can have a visualization port 720 at the distal end for providing an image back to a camera or optical viewfinder mounted at the proximal end of the bronchoscope 120. It is possible that the housing 850, when protruding from the distal end of the bronchoscope 120, can interfere with or obstruct the view through the visualization port 720. Therefore, it can be advantageous to configure the housing 850 so that it does not interfere with visualization through the visualization port 720, such as by moving the housing 850 as far away from the visualization port 720 as possible. FIG. 25 shows an embodiment of a housing 850 that is configured to not obstruct the visualization port 720. The housing 850 of FIG. 25 is mounted to the delivery catheter 110 in an eccentric or off-center manner.

In FIG. 25, the housing 850 is cylindrical, but mounted off-center relative to the catheter 110 relative to the longitudinal axis of the catheter 110. The visualization port 720 defines an axis 2510 that is aligned with the longitudinal axis of the bronchoscope 120 when laid straight. In one embodiment, the housing 850 has a size, shape, and position such that no portion of the housing 850 intersects the axis 2510 or such that the radial periphery of the housing 850 is at least 0.5 to 1 millimeters away from the axis 2510.

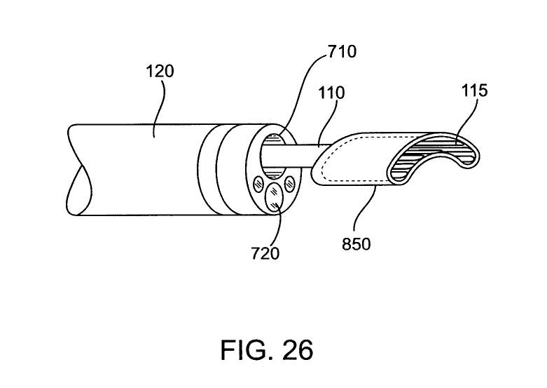

In another embodiment, shown in FIG. 26, the housing 850 has an irregular shape that is selected to not interfere with the visualization port 720. The housing 850 can have, for example, an oval, crescent, or half-circular cross-sectional shape which permits portions of the housing to be moved farther away from the visualization port than other portions via the visualization port. The housing shown in FIG. 26 can be used where the bronchial isolation device 115 can be compressed into a shape other than a cylinder. The housing can also be made of a malleable material so as to be shapeable by the user.

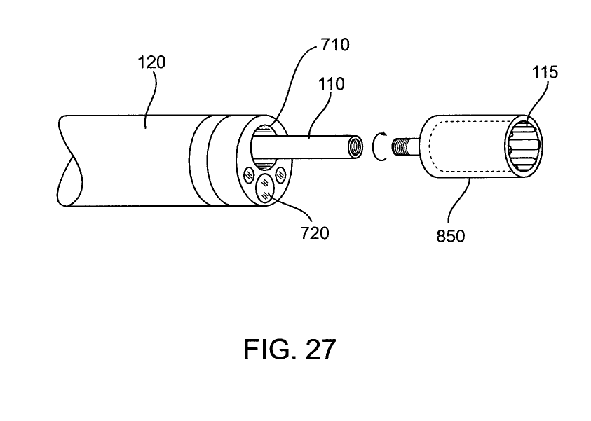

In another embodiment, shown in FIG. 27, the housing 850 is removably mounted to the distal end of the delivery catheter 110. In use, the distal end of the catheter 110 (with the housing 850 removed) is inserted into the working channel 710 via the working channel entry port 135. The catheter 110 is then advanced through the working channel 710 until the distal end of the catheter protrudes outward through the opening in the distal end of the working channel 710, as shown in FIG. 27. The removable housing 850 containing the compressed bronchial isolation device 115 is then attached to the distal end of the catheter 110. The attachment of the housing 850 to the catheter 110 can be accomplished in various manner, such as by clipping, screwing or other means. The inner member 825 is attached to the flange 910 within the housing by a ball and socket, snap, or other mechanism. The bronchoscope 110 with the delivery catheter 110 contained therein is then inserted into the bronchial tree of the patient and guided to the target bronchial lumen.

In another embodiment, the distal end of the catheter 110 can be deflected using a pull-wire attached to the distal end of the catheter 110. During use, the pull wire can be pulled to deflect the distal end of the catheter 110 so as to not obstruct visualization through the visualization port. Alternatively, a sleeve could be placed over the distal end of the catheter 110 such that the sleeve is shaped to deflect the catheter from obstructing the view through the visualization port.

### Housing Retraction Delivery System

As discussed above with reference to FIG. 11, one way to eject the bronchial isolation device 115 from the housing 850 is to slide the outer catheter member 820 in a proximal direction, while maintaining the inner catheter member 825 and flange 910 fixed. The proximal movement of the outer member 820 causes the attached housing 850 to also move in a proximal direction, while the flange 910 prevents the bronchial isolation device 115 from moving in the proximal direction. This results in the housing 850 sliding away from engagement with the bronchial isolation device 115 so that the bronchial isolation device 115 is released from the housing 850 and deployed. However, the aforementioned system will not work in the situation where the outer catheter member 820 must remain fixed relative to another object, such as the bronchoscope 120, an endotracheal tube, or the patient. Given that the outer catheter member 820 must move in the proximal direction to retract the housing 850, the position of the outer catheter member 820 cannot be fixed. There is now described a delivery catheter that overcomes this problem.

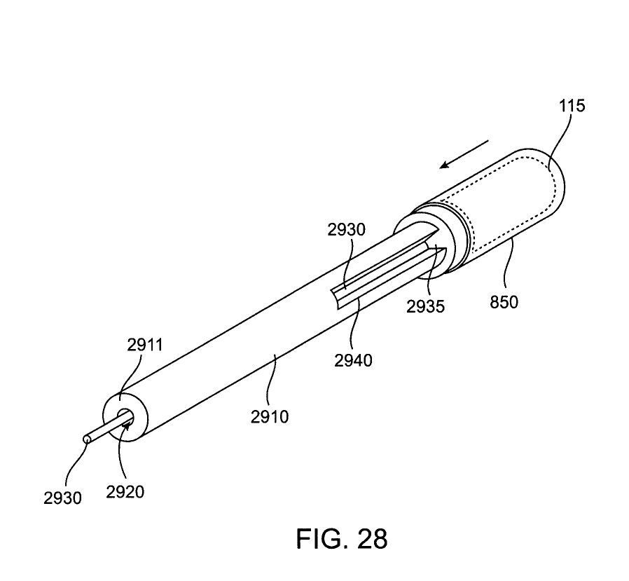

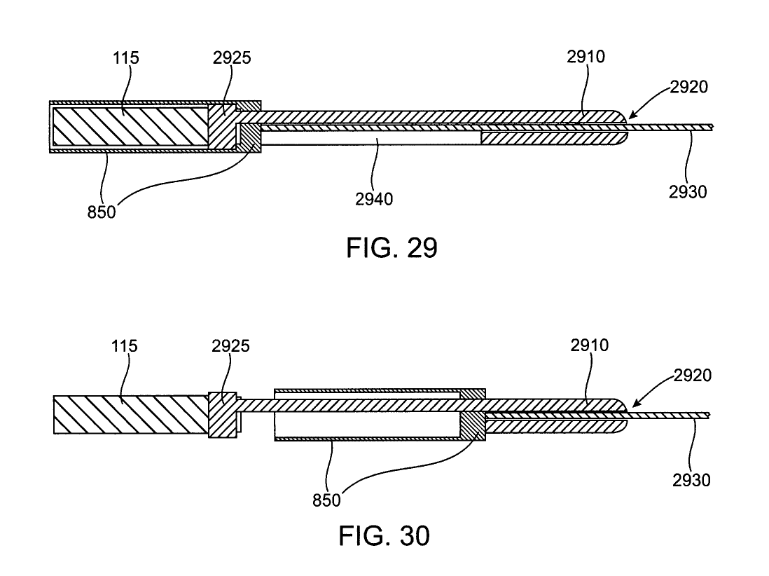

FIG. 28 shows a perspective view of the another embodiment of the delivery catheter, referred to as delivery catheter 2910, wherein the housing 850 is slidably mounted on the distal end of the catheter 2910. For illustration purposes, FIG. 28 shows the catheter 2910 cut at a location 2911, although it should be appreciated that the catheter 2910 actually extends to the handle at the proximal end. FIG. 29 shows a cross-sectional view of the distal region of the catheter 2910. The catheter 2910 comprises a single, elongate shaft that includes a center lumen 2920 that extends internally through the length of the catheter 2910. An ejection member comprising a flange 2925 (shown in FIG. 29) is fixedly or removably located at the distal end of the catheter 2910 inside the housing 850 and configured to engage the bronchial isolation device 115. The housing 850 is free to slide a predetermined distance along the length of the catheter 2910 such that the housing 850 can slide back and forth relative to the catheter 2910 and the flange 2925. A distal end of a retraction element, such as a pull wire 2930, is attached to the housing 850. The proximal end of the pull wire 2930 is attached to an actuator, such as a handle (not shown) located at the proximal end of the catheter 2910. The pull wire 2930 is at least partially positioned within the center lumen 2920 of the catheter 2910.

FIG. 29 shows the housing 850 in a distal-most position relative to the catheter 2910. The bronchial isolation device 115 is positioned within the housing 850 with a proximal edge of the bronchial isolation device 115 adjacent the flange 2925. Alternatively, the flange can be eliminated such that the bronchial isolation device 115 engages the distal end of the catheter 2910. The housing 850 can be retracted in a proximal direction relative to the catheter 2910 and the flange 2925 by pulling on the housing 850 using the pull wire 2930. In this regard, the proximal end of the pull wire can be attached to the handle at the proximal end of the catheter 2910. As discussed relative to the previous embodiments of the catheter, the handle 2910 can comprise two pieces, one of which is attached to the proximal end of the pull wire 2930 and the other of which is attached to the proximal end of the catheter 2910. Thus, the housing 850 can be retracted by moving the handle proximally to move the pull wire 2930 proximally. The pull wire 2930 thereby pulls the housing 850 in the proximal direction such that the shaft and the flange remain stationary relative to the bronchial wall while the housing moves and the device is deployed. Alternatively, if its not desired to fix the position of the housing relative to the bronchial wall, the housing can remain stationary and the wire used to push the flange and bronchial isolation device out of the housing. In such a case, the wire would have to be sufficiently rigid to provide a pushing force without bending.

FIG. 30 shows the housing 850 in a retracted state after the housing 850 has moved in the proximal direction. The housing 850 has moved proximally relative to the flange 2925 such that the flange 2925 is positioned outside of the housing 850. As the housing 850 moves proximally, the flange 2925 acts as a stop that prevents the bronchial isolation device 115 from also moving proximally. Alternatively, the distal end of the catheter 2910 can act as a stop if the flange is not used. Thus, the bronchial isolation device 115 is outside of the housing 850 when the housing 850 is in the retracted state.

In one embodiment, the housing 850 includes a guide element that interfaces with a corresponding guide element in the catheter 2910 for guiding the movement of the housing 850 relative to the catheter 2910 while the housing 850 is being retracted. As best shown in FIG. 28, 31, and 32, the housing guide element can comprise a guide tab 2935 that is slidably positioned within a corresponding elongate guide slot 2940 located in the distal end of the catheter 2910.

FIG. 31 shows an enlarged view of the housing 850 and distal region of the pull wire 2930. The guide tab 2935 is located at a proximal end of the housing 850. The guide tab 2935 extends radially so as to be attached to the pull wire 2930 at a central location of the housing 850.

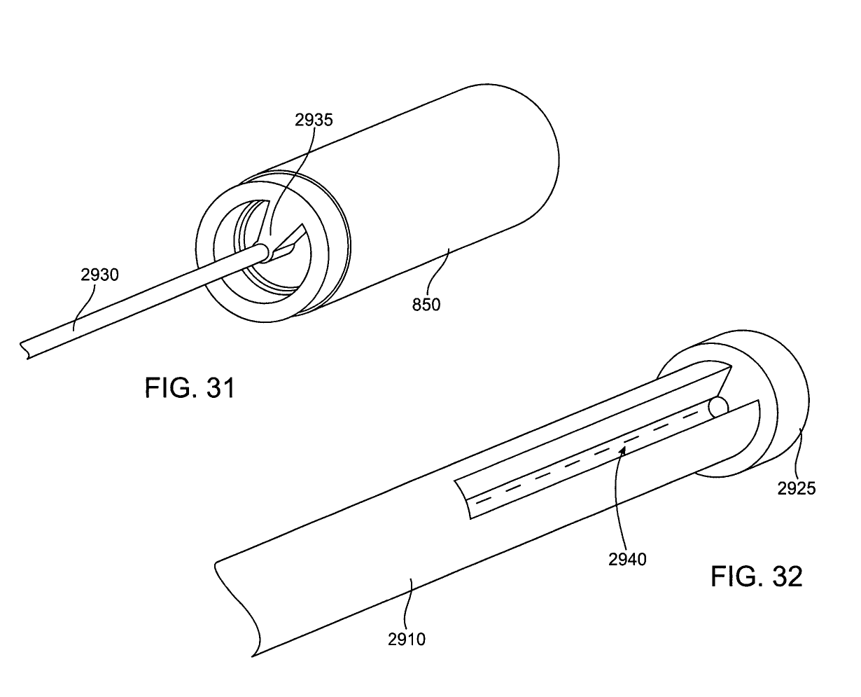

FIG. 32 shows an enlarged view of the distal region of the catheter 2910 without the housing 850. The flange 2925 is fixed to the distal end of the catheter 2910. The guide slot 2940 extends a predetermined distance along the length of the distal region of the catheter 2910. The guide slot 2940 is sized to slidably receive the guide tab 2935. When the housing 850 is retracted, the guide tab 2935 slides proximally within the guide slot 2940 up to the point at which the guide slot 2940 ends, at which point the guide tab 2935 will abut the proximal end of the guide slot 2940. In this manner, the guide tab 2935 acts as a detent to prevent the housing from being retracted beyond a predetermined distance. The interface between the guide tab 2935 and the guide slot 2940 provides for a smooth, controlled movement as the housing 850 moves in and out of the retracted state.

To assemble the catheter 2910, the housing 850 is slid onto the distal portion of the catheter 2910 and the flange 2925 is then attached to the distal end of the catheter 2910. The flange 2925 can be formed or attached with the housing 850 slid in a fully proximal location along the catheter 2910. The flange 2925 can be heat formed if the catheter material is a thermoplastic material, or can be a separately bonded or attached component. Depending on the configuration of the bronchial isolation device 115, the flange 2925 feature may not be needed in order to successfully deploy the device. If a flange 2925 is used, a sleeve can be added to the housing 850 to cover the guide slot 2940. The sleeve can retract with the housing 850 and can protect the guide slot 2940 and pull wire 2930 from damage. The housing 850 and/or sleeve may be formed from various materials, including metal or polymer.

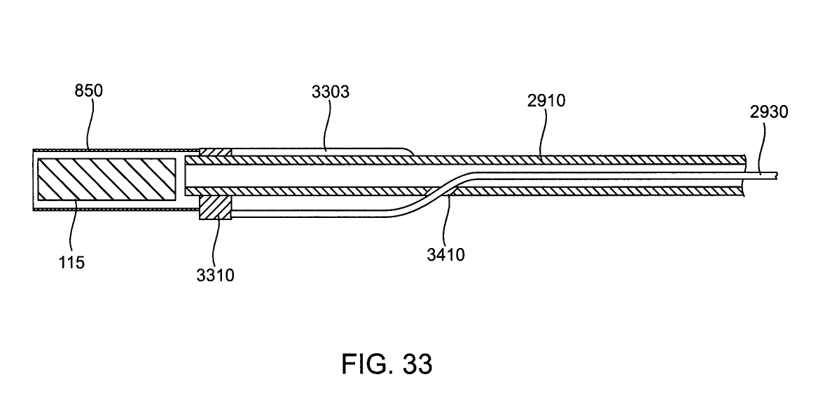

FIG. 33 shows a cross-sectional view of another embodiment of the catheter 2910 wherein the pull wire 2930 attaches to the radial periphery of the housing 850. The pull wire 2930 protrudes out of the catheter 2910 through an opening 3410, which is positioned in the distal region of the catheter 2910, such as just proximal to the proximal end of the guide slot. The distal region of the pull wire 2930 follows the outer surface of the catheter 2910 and the distal end of the pull wire 2930 is attached to the housing 850. It should be appreciated that more than one pull wire can be used. For example, two or more pull wires can be spaced around the periphery of the catheter 2910, such as where one wire is not sufficiently strong or where one wire leads to cocking of the housing 850 during deployment. The wire or wires can be attached to the housing in various ways, including welding or crimping using an outer crimp sleeve 3310, as shown in FIG. 33. Optionally, a wire protection sleeve 3303 can be added to the distal portion of the sliding housing to protect the exposed portion of the pull wire prior to device deployment.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

What is claimed:

1. An apparatus for deploying a bronchial device in a bronchial passageway in a lung of a patient, comprising:
a flexible shaft having an inner lumen and a distal end adapted to engage the bronchial isolation device;
a wire slidably disposed in the inner lumen; and
a housing movably coupled to the distal end of the shaft and configured to receive the bronchial device;
wherein the wire is connected to the housing to produce relative movement between the housing and the shaft to deploy the bronchial device from the housing.

2. The apparatus of claim 1, wherein the housing is releasably coupled to the shaft.

3. The apparatus of claim 1, further comprising a flange coupled to the distal end of the shaft and movably disposed in the housing, wherein the flange is adapted to engage the bronchial device.

4. The apparatus of claim 3, wherein the flange is releasably coupled to the shaft.

5. The apparatus of claim 1, wherein the housing is rigid.

6. The apparatus of claim 1, wherein the housing is shapable into a desired cross-sectional shape.

7. The apparatus of claim 1, wherein the housing has a non-circular cross-section.

8. The apparatus of claim 7, wherein the housing is crescent-shaped or partially-circular in cross-section.

9. The apparatus of claim 7, wherein the housing is oval or elliptical in cross-section.

10. The apparatus of claim 1, wherein the housing is configured to remain stationary relative to the bronchial passageway as the bronchial device is deployed therefrom.

11. The apparatus of claim 1, wherein the distal end of the shaft is configured to remain stationary relative to the bronchial passageway as the bronchial device is deployed from the housing.

12. The apparatus of claim 1, wherein the shaft is configured to be placed through a working channel of a bronchoscope.

13. The apparatus of claim 12, wherein the housing is collapsible for insertion through the working channel.

14. The apparatus of claim 12, wherein the housing has a cross-sectional size larger than a cross-sectional size of the working channel.

15. The apparatus of claim 12, wherein the housing is adapted for attachment to the shaft after the shaft has been positioned in the working channel.

16. The apparatus of claim 12, further comprising a handle adapted to move the wire relative to the shaft, the handle being adapted for attachment to the shaft and the wire after the shaft has been positioned in the working channel.

17. The apparatus of claim 12, further comprising a locking mechanism for locking the position of the shaft relative to the working channel.

18. The apparatus of claim 17, wherein the locking mechanism comprises a clamp slidably coupled to the shaft, the clamp being configured to engage the shaft for locking the shaft in a desired position.

19. The apparatus of claim 18, wherein the clamp may be coupled to the bronchoscope.

20. The apparatus of claim 1, wherein the shaft defines a longitudinal axis, the housing being coupled to the shaft off-center from the longitudinal axis.

21. A guidewire for guiding a bronchial device into a bronchial passageway in a lung, the bronchial device having an inner lumen, the guidewire comprising:
an elongate flexible wire having a distal end configured for introduction through the patient's trachea into the bronchial passage, the wire being slidably positionable in the inner lumen of the bronchial device;
an anchor coupled to the wire, the anchor being configured to engage a wall of the bronchial passage to retain the position of the guidewire therein.

22. The guidewire of claim 21, wherein the bronchial device comprises a bronchoscope.

23. The guidewire of claim 21, wherein the bronchial device comprises a delivery catheter for delivering a flow control device into the bronchial passage.

24. The guidewire of claim 21, wherein the bronchial device comprises a flow control device for modifying fluid flow through the bronchial passage.

25. The guidewire of claim 21, wherein the anchor comprises a plurality of struts, the struts being movable radially outward from the wire to engage the wall of the bronchial passage.

26. The guidewire of claim 25, wherein the wire has an inner lumen, the guidewire further comprising an inner wire slidably disposed in the inner lumen for moving the struts.

27. The guidewire of claim 21, wherein the anchor comprises an inflatable balloon, the wire having an inflation lumen therein in communication with the balloon.

28. A method of positioning a device in a bronchial passageway in a patient's lung, the method comprising:
positioning a guidewire in the bronchial passageway, the guidewire having an anchor coupled thereto;
engaging the anchor to a wall of the bronchial passage to retain the position of the guidewire therein;
inserting the guidewire through a lumen in the device; and
sliding the device along the guidewire into the bronchial passage.

29. The method of claim 28, wherein engaging the anchor to a wall of the bronchial passageway comprises expanding the anchor to engage the wall of the bronchial passageway.

30. The method of claim 29, wherein expanding the anchor comprises inflating a balloon coupled to the guidewire.

31. The method of claim 29, wherein expanding the anchor comprises moving a plurality of struts radially outward from the guidewire.

32. The method of claim 28, wherein the device comprises a bronchoscope.

33. The method of claim 28, wherein the device comprises a delivery catheter.

34. The method of claim 28, further comprising deploying a flow control element into the bronchial passageway from the device.

35. The method of claim 28, wherein positioning the guidewire in the bronchial passageway comprises positioning the guidewire in a lumen of a bronchoscope positioned in the bronchial passageway.

36. The method of claim 35, further comprising removing the bronchoscope after engaging the anchor to leave the guidewire in place in the bronchial lumen.

37. A method of deploying a bronchial device in a bronchial passageway in a patient's lung, the method comprising:
positioning a bronchoscope in the patient's lung, the bronchoscope having a working channel;
positioning a shaft of a delivery catheter through the working channel;
coupling a housing to a distal end of the shaft while the shaft is positioned in the working channel;
advancing the delivery catheter with the housing carrying the bronchial device until the housing is positioned in the bronchial passageway; and
releasing the bronchial device from the housing.

38. The method of claim 37, further comprising inserting the bronchial device into the housing prior to coupling the housing to the distal end of the shaft.

39. A method of deploying a bronchial device in a bronchial passageway in a patient's lung, the method comprising:
providing a bronchoscope, the bronchoscope having a working channel;
positioning a shaft of a delivery catheter through the working channel, the shaft having a housing coupled to its distal end, the housing carrying the bronchial device;
coupling a handle to a proximal end of the shaft with the shaft positioned in the working channel, the handle having a movable actuator;
positioning the bronchoscope in the patient's lung with the shaft positioned in the working channel;
advancing the delivery catheter until the housing is positioned in the bronchial passageway; and
moving the actuator to release the bronchial device.

40. A method of deploying a bronchial device in a bronchial passageway in a patient's lung, the method comprising:
positioning a bronchoscope in the patient's lung, the bronchoscope having a working channel;
positioning a delivery catheter through the working channel, the delivery catheter having a housing carrying the bronchial device;
advancing the delivery catheter until the housing is positioned in the bronchial passageway;
locking the delivery catheter in position relative to the working channel; and
releasing the bronchial device from the housing.
